(19) United States
(12) Patent Application Publication
(10) Pub. No.: US 2025/0332031 A1
(43) Pub. Date: Oct. 30, 2025

(54) APPARATUS, SYSTEM, AND METHOD FOR REMOVING CERUMEN

(71) Applicant: BriBev Holdings, Inc., London (CA)

(72) Inventor: Brian Rotenberg, London (CA)

(73) Assignee: BriBev Holdings, Inc., London (CA)

(21) Appl. No.: 19/063,094

(22) Filed: Feb. 25, 2025

Related U.S. Application Data
(60) Provisional application No. 63/638,380, filed on Apr. 24, 2024.

(51) Int. Cl. A61F 11/00

(52) U.S. Cl. CPC A61F 11/006

(57) ABSTRACT

An apparatus, system, and method for removing cerumen from a subject's ear is disclosed. The apparatus includes a tip having a shaft with a passage extending from a proximal end and a balloon positioned away from the proximal end. The balloon is coupled to the shaft in fluid communication with the passage. The apparatus further includes a body comprising a head configured to couple to the shaft and a handle attached to the head for user manipulation. An inflator configured to introduce a fluid into the passage to inflate the balloon. The apparatus includes safety features such as an ergonomic ambidextrous handle to enable use with either hand, a stop to prevent eardrum damage, a pressure-regulating mechanism to prevent overinflation, a controlled deflation system, and a soft, flexible balloon material to minimize discomfort or injury. These features ensure safe and effective cerumen removal by dislodging earwax without excessive pressure.

### CROSS-REFERENCE TO RELATED APPLICATIONS

This application claims the benefit of U.S. Provisional Application No. 63/638,380, filed Apr. 24, 2024, which is hereby incorporated by reference in its entirety.

### TECHNICAL FIELD

The present disclosure relates to surgical devices, systems, instruments, and methods. More specifically, the present disclosure relates to patient-specific instruments, instruments, and/or methods for removing cerumen from a subject, such as a patient.

### BACKGROUND

The removal of cerumen (earwax) has presented challenges throughout history, as excessive accumulation can lead to discomfort, impaired hearing, and an increased risk of ear infections (otitis externa and media). Past civilizations employed rudimentary tools such as metal scoops or feathers to extract cerumen, often resulting in pain and injury due to the delicate nature of the ear canal. Over time, healthcare practitioners developed more advanced methods, including irrigation techniques and suction-based extraction, but these methods require precise control to avoid complications such as perforation of the tympanic membrane (eardrum) or damage to the sensitive epithelial lining of the auditory canal. Failure to remove cerumen regularly can contribute to conductive hearing loss, a sensation of fullness or pressure in the ear, and even dizziness (vertigo), as cerumen impaction can interfere with normal auditory and vestibular function.

Efforts to remove cerumen safely remain a significant concern, particularly due to the risks associated with self-removal techniques employed by users/subjects/patients. Many individuals attempt to extract cerumen using cotton swabs, ear candles, or improvised tools, which can inadvertently push the wax deeper into the canal, worsening impaction and potentially causing abrasions, infections, or eardrum perforation. Even medically approved methods, particularly when self-administered, such as irrigation with warm water or hydrogen peroxide solutions, carry risks of ear infections, vertigo, and temporary hearing disturbances if not administered correctly. Improper self-removal techniques can result in direct injury to the tympanic membrane or surrounding structures, leading to pain, temporary or permanent hearing loss, and the need for medical intervention. Additionally, individuals with narrow ear canals, excessive cerumen production, or preexisting conditions such as aural eczema or aural psoriasis may experience heightened sensitivity and increased risk of complications. Consequently, there remains a need for improved cerumen removal technologies that provide safe, effective, patient-friendly, available for self-administration solutions while minimizing discomfort and reducing the risks of self-inflicted injury.

### SUMMARY

The various apparatus, devices, systems, and/or methods of the present disclosure have been developed in response to the present state of the art, and in particular, in response to the problems and needs in the art that have not yet been fully solved by currently available technology.

A cerumen removal apparatus is provided designed for effective earwax extraction while maintaining safety and user comfort. The apparatus features a tip that includes a shaft with an internal passage extending from its proximal end. Positioned distally along the shaft is a balloon, which is fluidly connected to the passage. The balloon's controlled inflation facilitates cerumen extraction by bypassing the cerumen during insertion, then after inflation engaging the auditory canal walls to push the cerumen out of the ear canal as the balloon is extracted.

The body of the apparatus includes a head that connects to the shaft, a handle designed for ease of manipulation, and an inflator that introduces fluid into the passage to inflate the balloon. The tip is replaceable, attached to the body through a secure attachment mechanism, such as a luer lock. The body may further include a neck extending from the head, which connects to the shaft. To enhance usability, the handle incorporates an ergonomic, ambidextrous grip that accommodates multiple fingers and may include a dedicated thumb grip for additional control.

To ensure user safety, the design includes a stop positioned at the distal end of the neck to limit insertion depth and prevent contact with the tympanic membrane. The inflator includes a chamber storing the fluid, with an outlet providing controlled flow into the passage. A driver mechanism, such as a piston, facilitates fluid movement by pressurizing the chamber. In certain configurations, the inflator is a syringe with a barrel containing fluid, a plunger that extends proximally from the body, and a threaded engagement between the plunger and the barrel for controlled inflation. The balloon may include a textured surface to enhance cerumen adhesion during extraction.

An alternative implementation integrates a syringe directly into the body, positioning the shaft to extend outward. The handle in this configuration supports a four-finger grip for stability. The balloon is designed to have a smaller cross-sectional diameter than the auditory canal when deflated but expands upon inflation to engage the canal surface. A locking mechanism may be included to maintain balloon inflation for consistent cerumen removal.

A method is provided for using the device to remove cerumen from an ear. The process involves grasping the apparatus, inserting the balloon into the auditory canal until the stop engages the ear, inflating the balloon to mobilize cerumen, and withdrawing the balloon while scraping cerumen toward the canal's proximal opening. Additional steps may include deflating the balloon, cleaning the extracted cerumen, and reusing or exchanging the tip. The method may also be adapted for self-use or specialized conditions requiring different tip configurations.

These implementations ensure a safe, controlled, and effective approach to cerumen removal while prioritizing user convenience and patient safety.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings.

FIG. 1 is a block diagram illustrating one embodiment of a system for removing cerumen from a subject/patient according to one embodiment.

FIG. 2A is a block diagram illustrating one embodiment of a driver for use in removing cerumen from a subject/patient according to one embodiment.

FIG. 2B is a block diagram illustrating one embodiment of a driver for use in removing cerumen from a subject/patient according to one embodiment.

FIG. 3A is a perspective view of one embodiment of an apparatus for removing cerumen from a subject/patient according to one embodiment.

FIG. 3B a cross-section view illustrating a view along lines 3B/6-3B/6 of FIG. 3A, according to one embodiment of an apparatus for removing cerumen from a subject/patient.

FIGS. 4A-4B are perspective views of parts of an apparatus for removing cerumen from a subject/patient according to one embodiment.

FIG. 5A is a side view of an inflator according to one embodiment.

FIG. 5B is an exploded view of an inflator according to one embodiment.

FIG. 6 is a cross-section view illustrating a view along lines 3B/6-3B/6 of FIG. 3A, according to one embodiment.

FIG. 7 is a cross-section view illustrating a view along lines 3B/6-3B/6 of FIG. 3A, according to another embodiment.

FIG. 8 is a flowchart diagram depicting a method for removing cerumen, according to one embodiment.

### DETAILED DESCRIPTION

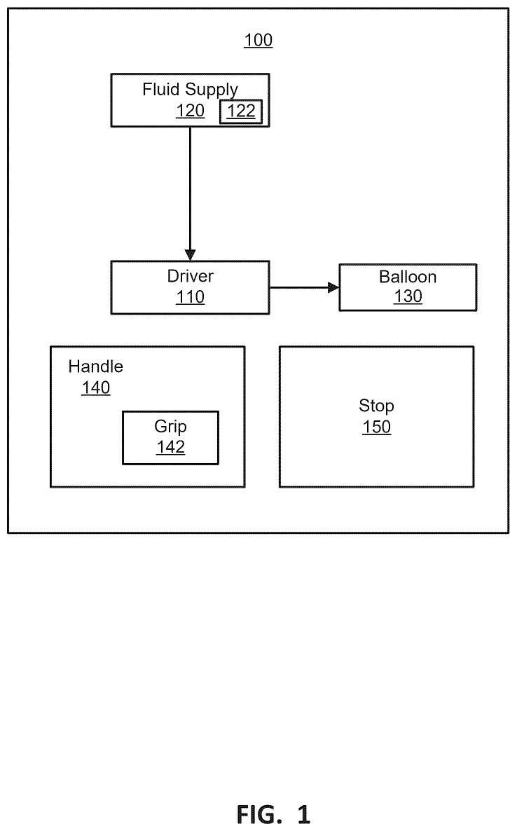

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and/or method, as represented in FIGS. 1 through 8, is not intended to limit the scope of the disclosure but is merely representative exemplary of exemplary embodiments.

“Balloon” refers to a flexible or pliable or resilient structure capable of containing a fluid. Typically, a balloon includes an opening to permit fluid to enter and/or exit the balloon. In certain embodiments, a balloon may include two or more openings which can be used separately or together to permit fluid to enter and/or exit the balloon. A balloon can be made from a variety of materials, including plastic, silicone, rubber, latex, and similar materials. Examples of a balloon include, but are not limited to, a bubble, a bladder, an inflatable member, and similar items.

“Fluid Supply” refers to an apparatus, instrument, structure, member, device, component, system, or assembly structured, organized, configured, designed, arranged, or engineered to store, retain, or hold a fluid. In certain embodiments, a fluid supply include a plurality of components coupled to, connected to, and/or integrated with the fluid supply. In certain embodiments, a fluid supply is an open container. In one embodiment, a fluid supply is a closed, sealed container. In one embodiment, a fluid supply includes a lid or cap that enables the fluid supply to operate either open or closed and/or sealed. In certain embodiments, a fluid supply can include a plurality of containers or chambers. A fluid supply may include one or more openings configured to enable fluid to pass into and/or out of the fluid supply. In certain embodiments, the one or more openings may include one or more valves that control flow of the fluid through a corresponding opening.

As used herein, an “opening” refers to a gap, a hole, an aperture, a port, a portal, a slit, a space or recess in a structure, a void in a structure, or the like. In certain embodiments, an opening can refer to a structure configured specifically for receiving something and/or for allowing access. In certain embodiments, an opening can pass through a structure. In such embodiments, the opening can be referred to as a window. In other embodiments, an opening can exist within a structure but not pass through the structure. In other embodiments, an opening can initiate on a surface or at an edge or at a side of a structure and extend into the structure for a distance, but not pass through or extend to another side or edge of the structure. In other embodiments, an opening can initiate on a surface or at an edge or at a side of a structure and extend into the structure until the opening extends through or extends to another side or edge of the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape. As used herein, the term “opening” can include one or more modifiers that define specific types of “openings” based on the purpose, function, operation, position, or location of the “opening.” As one example, a “fastener opening” refers to an “opening” adapted, configured, designed, or engineered to accept or accommodate a “fastener.”

“Port” refers to an opening configured, engineered, and/or designed for passage of a fluid. Often “port” is used with a modifier describing and differentiating a direction of flow of a fluid through the port. For example, an “inlet port” refers to a port configured and/or oriented to allow a fluid to pass through the port and into a container or other vessel. An “outlet port” refers to a port configured and/or oriented to allow a fluid to pass through the port and out of a container or other vessel. Similarly, “port” can be used with a modifier that identifies the container or vessel to which the port enables fluid communication of the fluid. For example, a “reservoir inlet port” refers to a port that enables a fluid to pass into a reservoir. Similarly, a “reservoir outlet port” refers to a port that enables a fluid to pass out of a reservoir.

“Fluid” refers to a substance that flows and is capable of flowing and can easily or readily take the shape of a container. Fluid is characterized by its ability to deform continuously under an applied shear stress or force, regardless of the magnitude of the stress or force. Examples of fluids can include liquids, such as water, oil, and alcohol, as well as gases, such as air, nitrogen, and oxygen. These substances lack a fixed shape and can flow in response to applied forces. (© ChatGPT 3.5 Version, Modified, accessed chat.openai.com/chat Apr. 9, 2024).

“Textured Surface” refers to a surface or other feature of an object, instrument, or apparatus. The textured surface may abut, touch, or contact a surface of a subject or patient such as skin or surfaces of another anatomical structure (e.g., the external auditory canal). In another aspect, a textured surface may include projections and recesses across at least a portion of a surface. A textured surface may be uniform, contoured, or distributed on an outside of an object according to a pattern.

“Screw Thread” refers to a continuous helical ridge, typically of uniform cross-section. A screw thread may be formed on an internal or an external surface of a cylindrical or conical object. A screw thread can serve to convert between rotational and linear motion or force and/or to securely fasten two objects together. (© ChatGPT 40 Version, Modified, accessed chat.openai.com Jan. 28, 2025)

“Subject” refers to an individual—human or animal—that is the focus or recipient of an intervention, observation, treatment, procedure, medical procedure, or experimental procedure. (© ChatGPT 40 Version, Modified, accessed chat.openai.com Jan. 28, 2025)

“Cerumen” or “Earwax” refers to a naturally occurring substance produced by glands in the ear canal. Cerumen typically includes a mixture of secretions from ceruminous glands and sebaceous glands, along with dead skin cells. Cerumen serves several functions, including lubricating the ear canal, protecting the ear from foreign particles, such as dust and debris, and helping to prevent infection by trapping bacteria and other pathogens. While cerumen is typically beneficial, excessive accumulation can lead to blockages or impactions, potentially causing discomfort or hearing problems. (© ChatGPT 3.5 Version, Modified, accessed chat.openai.com/chat Apr. 9, 2024).

As used herein, a “shaft” refers to a long narrow structure, device, component, member, system, or assembly that is structured, organized, configured, designed, arranged, or engineered to support and/or connect a structure, device, component, member, system, connected to each end of the shaft. A shaft can be hollow or solid. Typically, a shaft is configured to provide rigid support and integrity in view of a variety of forces including tensile force, compression force, torsion force, shear force, and the like. In addition, a shaft can be configured to provide rigid structural support and integrity in view of a loads including axial loads, torsional loads, transverse loads, and the like. A shaft may be oriented and function in a variety of orientations including vertical, horizontal, or any orientation between these and in two or three dimensions. A shaft may be made from a variety of materials including, but not limited to, metal, plastic, ceramic, wood, fiberglass, acrylic, carbon, biocompatible materials, biodegradable materials or the like. A shaft may be formed of any biocompatible materials, including but not limited to biocompatible metals such as Titanium, Titanium alloys, stainless steel, carbon fiber, combinations of carbon fiber and a metallic alloy, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others, or any combination of these materials.

“Auditory canal” or “Ear canal” refers to a tube-like structure that extends from the outer ear to the middle ear. The ear canal serves as a passageway for sound waves to travel from the external environment to the eardrum (tympanic membrane). The ear canal is lined with skin and contains tiny hairs and glands that produce cerumen (earwax). The ear canal's shape and structure help to amplify and direct sound waves towards the eardrum, where they are then transmitted to the middle ear for further processing. The “auditory canal” or “Ear canal” may also be referred to as the “external auditory canal” or the “acoustic meatus”. (© ChatGPT 3.5 Version, Modified, accessed chat.openai.com/chat Apr. 9, 2024).

As used herein, a “driver” refers to a mechanical piece, component, or structure for imparting motion to another piece, component, or structure. (“driver.” Merriam-Webster.com. Merriam-Webster, 2021. Web. 6 Jan. 2021. Modified.) In certain embodiments, a driver can be a wheel configured or connected to other parts such that rotation or motion of the driver causes motion of other interconnected or intercoupled parts of a component, system, apparatus, or device.

As used herein, a “driver” refers to a component, structure, device, system, apparatus, or mechanical piece for imparting motion or force on to another piece, aspect, component, or structure. (“driver.” Merriam-Webster.com. Merriam-Webster, 2021. Web. 6 Jan. 2021. Modified.) A driver may impart a force or cause a motion on a mechanical part or set of parts or on a fluid such as a liquid or gas. Where the driver imparts a motion, the motion cause translation, rotation, and/or both translation and rotation. Where the driver imparts a force on a fluid, the driver may increase pressure on a fluid and/or the driver may decrease pressure on a fluid. In certain embodiments, a driver can be a wheel configured or connected to other parts such that rotation or motion of the driver causes motion of other interconnected or intercoupled parts of a component, system, apparatus, or device.

As used herein, a “handle” refers to a structure used to hold, control, or manipulate a device, apparatus, component, tool, or the like. A “handle” may be designed to be grasped and/or held using one or two hands of a user. In certain embodiments, a handle may be sized and/or configured to accommodate two or more fingers of a user. Alternatively, or in addition, in certain embodiments, a handle may include a grip. In certain embodiments, a handle may be an elongated structure. In one embodiment, a handle may be in the form of a knob and may be a short stubby structure.

“Contour” refers to an outline representing or bounding a shape or form of an object. Contour can also refer to an outside limit of an object, area, or surface of the object. (Search “contour” on wordhippo.com. WordHippo, 2023. Web. Modified. Accessed 13 Jun. 2023.)

“Grip” refers to a structure configured to accommodate and/or enable stable and secure engagement between a hand of a user and another object. A grip is configured to enable a user to grasp or hold an object and enable controlled, stable, control and/or manipulation of the object. (© ChatGPT 3.5 Version, Modified, accessed chat.openai.com/chat Apr. 9, 2024). In certain embodiments, a grip can include one or more structures to accommodate at least one or more fingers of a user. In certain embodiments, a grip may be configured to accommodate one or more fingers and one or more other parts of a hand. For example, a grip may include a palm grip configured to accommodate and engage with a palm of a hand of a user as the user uses an object. Similarly, a grip may include thumb grip to accommodate and engage with a thumb of the hand of the user as the user controls, handles, or manipulates the object. In certain embodiments, a grip (e.g., palm grip, thumb grip, finger grip, or the like) may comprise a contoured surface of a structure such as a handle.

“Finger grip” refers to a grip configured specifically to accommodate a finger of a user and/or a particular finger of a user, such as an index finger or a thumb. In certain embodiments, a finger grip may comprise a contoured surface of a structure such as a handle.

As used herein, a “stop” refers to an apparatus, instrument, structure, member, device, component, system, or assembly structured, organized, configured, designed, arranged, or engineered to prevent, limit, impede, stop, or restrict motion or movement and/or operation of another object, member, structure, component, part, apparatus, system, or assembly.

Standard medical planes of reference and descriptive terminology are employed in this disclosure. While these terms are commonly used to refer to the human body, certain terms are applicable to physical objects in general.

A standard system of three mutually perpendicular reference planes is employed. A sagittal plane divides a body into right and left portions. A coronal plane divides a body into anterior and posterior portions. A transverse plane divides a body into superior and inferior portions. A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure. Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure.

Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

“Tympanic membrane”, also referred to as an ear drum, refers to structure in the middle ear of a person or animal. The structure is a thin, semitransparent membrane located in the middle ear. The ear drum separates the external ear canal from the middle ear cavity. The tympanic membrane/ear drum plays a role in the transmission of sound waves. When sound waves enter the ear canal, they cause the tympanic membrane to vibrate. These vibrations are then transmitted through the middle ear bones to the inner ear, where they are converted into nerve impulses that are interpreted by the brain as sound. (© ChatGPT 3.5 Version, Modified, accessed chat.openai.com/chat Apr. 9, 2024)

“Inflator” refers to a structure, device, component, member, system, assembly, and/or part configured inflate or cause inflation of a structure, system, device, or apparatus. Generally, an inflator is configured to drive a fluid such as a liquid or a gas into a closed or closable system such that the system receives more fluid or one or more fluids within the system have an increase in pressure within the system. An inflator can be a single component or structure or a plurality of components or structures or an assembly. The inflator can be mechanical, pneumatic, hydraulic, manual, chemical, thermal, pyrotechnic, electronic, or electromechanical.

As used herein, “actuator” refers to a component of a machine, component, structure, device, or assembly that initiates or causes action such as motion and/or control of a component, structure, lever, mechanism, or system. (Search “actuator” on Wikipedia.com Nov. 15, 2021. CC-BY-SA 3.0 Modified. Accessed Dec. 28, 2021.) In certain embodiments, an actuator may comprise a mechanical part, structure and/or device. In certain embodiments, an actuator may comprise a switch, a lever, a knob, a dial, or the like.

“Deactuator” refers to a component of a machine, component, structure, device, or assembly that reverses or causes a reverse or opposite action such as motion and/or control and an actuator. In certain embodiments, a deactuator may comprise a mechanical part, structure and/or device. In certain embodiments, a deactuator may comprise a switch, a lever, a knob, a dial, or the like.

As used herein, a “lock” or “lock mechanism” refers to an object, member, structure, component, part, apparatus, system, or assembly that either alone or in combination with other parts or components prevents, limits, impedes, is in a fixed relationship to, stops, or restricts motion or movement and/or operation of the another object, member, structure, component, part, apparatus, system, or assembly.

“Tip” refers to an end of a structure. Often, a tip includes tapered edges that come together to form an edge and/or a point. A tip can be sharp or blunt. A tip can have a variety of shapes, sizes, and cross sections. A tip can have a circular cross section. A tip can include a single structure or a plurality of structures such as in a system or an assembly.

As used herein, “passage” refers to a duct, a vessel, an opening, a void, or other channel in a body, part, component, or structure, of a an apparatus, instrument, structure, member, device, component, system, or assembly. In certain embodiments, a passage is narrow and longer than the passage is wide. (Search “passage” on wordhippo.com. WordHippo, 2021. Web. Accessed 15 Nov. 2021. Modified.)

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like. In one embodiment, a body is a housing. In another embodiment, a housing is a body.

As used herein, a “housing” refers to a structure that serves to connect, interconnect, surround, enclose, and/or protect one or more other structural components. A housing may be made from a variety of materials including, but not limited to, metal, plastic, ceramic, wood, fiberglass, acrylic, carbon, or the like. Often a housing is made from plastic due to its lower expense, strength, and durability. A housing may also be formed of any materials, including but not limited to metals such as Aluminum, Steel, Carbon Fiber, Titanium, Titanium alloys, stainless steel alloys, cobalt-chromium steel alloys, nickel-titanium alloys, shape memory alloys and/or others. A housing may include a frame or framework or function within a larger system, component, structure, or device.

“Head” refers to a device, apparatus, member, component, system, assembly, module, subsystem, circuit, or structure, organized, configured, designed, arranged, or engineered to have a more prominent role in a particular feature, design, structure, function, operation, process, method, and/or procedure for a device, apparatus, member, component, system, assembly, module, subsystem, circuit, or structure the includes, is coupled to, or interfaces with the head. In certain embodiments, the head may sit at the top or in another prominent position when interfacing with and/or coupled to a device, apparatus, member, component, system, assembly, module, subsystem, circuit, or structure.

“Neck” refers to a device, apparatus, member, component, system, assembly, module, subsystem, circuit, or structure, that connects two other parts, structures, and/or components. Often a neck is a structure that connects to a head. In certain embodiments, a neck may connect a head structure to a body or trunk structure. In certain embodiments, a neck is a narrow structure in relation to other structures of a device, apparatus, member, component, system, assembly, module, subsystem, circuit, or structure.

“Outlet” refers to a point or opening through which something, such as electricity, water, air, goods, or a fluid, can pass or be distributed. In general, an outlet serves as a point of distribution or dispersion for something, allowing it to flow or be accessed. (© ChatGPT 3.5 Version, Modified, accessed chat.openai.com/chat Apr. 9, 2024).

“Piston” refers to a component typically found in engines, compressors, pumps, and hydraulic systems. Often, a piston is cylindrical. In certain embodiments, a piston may move back and forth within a cylinder or other chamber in response to pressure or mechanical force. Pistons are often attached to a connecting rod, and the movement of a piston may convert pressure into mechanical energy or mechanical motion, or vice versa. (© ChatGPT 3.5 Version, Modified, accessed chat.openai.com/chat Apr. 9, 2024)

“Rod” refers to an instrument, structure, device, or component that is long and slender or narrow and structured, organized, configured, positioned, designed, arranged, and/or engineered to press or push against or couple to another structure, instrument, component, or device.

“Plunger” refers to a cylindrical or tapered mechanism or structure typically used to create or control fluid pressure, motion, or displacement within a closed system. A plunger may include a rod or shaft with a larger diameter head or disc at one end, often referred to as the “plunger head.” Plungers are commonly used in various applications, including plumbing, hydraulic systems, pumps, and syringes. (© ChatGPT 3.5 Version, Modified, accessed chat.openai.com/chat Apr. 9, 2024)

In plumbing, a plunger is a tool consisting of a handle attached to a rubber suction cup. A plunger is used to create a vacuum or pressure differential within a pipe or drain, helping to dislodge clogs by forcing air or water through the system. In hydraulic systems, a plunger is a component that moves back and forth within a cylinder, often driven by hydraulic pressure, to exert force or control the flow of fluids. In syringes, a plunger is a part that fits into a barrel and is pushed or pulled to expel or draw in fluids. (© ChatGPT 3.5 Version, Modified, accessed chat.openai.com/chat Apr. 9, 2024).

“Wall” refers to a structure that serves to separate one part or area of a structure from another. A wall may also serve as a barrier that prevents passage of fluid or solids between two parts or areas of a device or structure. A wall can also serve to define a boundary between one or more sections of a structure or device. For example, a wall can define an interior chamber or room and/or an exterior chamber, area, or room.

“Compression force”, also known as compressive force, refers to a type of mechanical force that acts to decrease the volume of an object or material by pushing its particles closer together. A compression force may be exerted uniformly in all directions on the object or material. Alternatively, a compression force may be exerted on a side or wall of an object or set of material. Compression forces can be generated when an external load or pressure is applied to an object, or part of an object causing the object, or part of the object to undergo compression or deformation. (© ChatGPT 3.5 Version, Modified, accessed chat.openai.com/chat Apr. 9, 2024).

“Syringe” refers to is a medical device used for injecting or withdrawing fluids. A syringe can include several basic parts: a barrel, a plunger, a needle, a tip/cap, a luer lock/luer slip, plunger seal/gasket, and/or a flange.

A barrel is a main body of the syringe, usually made of plastic or glass, which holds a fluid to be injected or withdrawn. The barrel may include volume markings to indicate the amount of fluid inside, allowing for accurate measurement.

A plunger is a movable component that fits snugly inside the barrel. The plunger usually has a handle or thumb grip at one end for easy manipulation. When the plunger is pushed or pulled, the plunger creates a vacuum or pressure within the barrel, allowing the fluid to be drawn into the syringe or expelled from the syringe.

The needle is a hollow, pointed metal tube attached to the end of the syringe barrel. A needle can be used to penetrate the skin or other tissues for an injection or withdrawal of fluids. Needles come in various lengths and gauges depending on the specific application. In certain embodiments, a syringe may not include a needle and instead may be coupled to another structure such as a fluid passage.

In certain embodiments, a syringe may include a tip of the syringe where the needle can be attached. Some syringes may come with a removable cap to cover a needle before use. In certain embodiments, a syringe may include a luer lock or luer slip. These are two common types of connections used to attach a needle to the syringe. The luer lock mechanism involves twisting a needle onto the syringe for a secure connection, while the luer slip mechanism involves simply pushing a needle onto the syringe.

A plunger seal or gasket is a rubber or silicone ring located at the base of the plunger. The plunger seal or gasket forms a tight seal against the inner walls of a barrel, preventing leakage of the fluid. Some syringes have a flange, which is a flat, wing-like extension at the base of the barrel. The flange provides stability and support when the syringe is held or manipulated. (© ChatGPT 3.5 Version, Modified, accessed chat.openai.com/chat Apr. 9, 2024).

In certain embodiments, the syringe couples to a fluid passage such as a hollow opening of a shaft rather than coupling to a needle. As with a needle, the syringe may couple to a shaft using a luer lock or a luer slip.

“Depressed state” refers to a state or condition in which a device, structure, component, apparatus, system, and/or assembly has been pressed from a first position to a second position. Often, a depressed state includes a state in which the device, structure, component, apparatus, system, and/or assembly is moved from an extended position to a non-extended position.

As used herein, a “spring” or “biasing member” refers to an elastic structure that stores mechanical energy. Springs can be made of a variety of elastic material such as spring steel or plastic and can be cylindrical and/or helical in shape. Various types of springs can be used including coil springs, torsion springs, flat springs, and the like. (Search “spring (device)” on Wikipedia.com Nov. 28, 2020. Modified. Accessed Jan. 6, 2020.)

“Biasing force” refers to a force applied to a mechanical system, structure, or component to maintain or adjust its position, orientation, or behavior in a desired manner. A biasing force is often used to counteract other forces or influences acting on the system and ensure its stable operation under varying conditions.

A biasing force may be encountered in various mechanisms, such as springs, weights, magnets, or pneumatic or hydraulic systems. These components apply a force or torque to maintain equilibrium, provide resistance, or produce a predetermined response when the system is subjected to external loads or disturbances. (© ChatGPT 3.5 Version, Modified, accessed chat.openai.com/chat Apr. 9, 2024).

“Tube” refers to any structure or component that is hollow and cylindrical in shape. (Search “tube” on wordhippo.com. WordHippo, 2022. Web. Accessed 21 Jul. 2022. Modified.)

“Tragus” refers to a small, pointed, cartilaginous projection located on the outer ear, just in front of the ear canal and partially covering ear canal. The tragus is situated opposite the antitragus, another small projection on the ear. The tragus helps to funnel sound waves into the ear canal and also aids in protecting the ear canal from foreign objects. The tragus is a feature of the anatomy of the human ear and varies in size and shape among individuals. (© ChatGPT 3.5 Version, Modified, accessed chat.openai.com/chat Apr. 9, 2024).

“Antitragus” refers to a small projection of cartilage located on the external ear, positioned opposite and superior to the earlobe. (© ChatGPT 3.5 Version, Modified, accessed chat.openai.com/chat Apr. 9, 2024).

As used herein, a “condition” refers to a state of something with regard to its appearance, state, quality, or working order. In certain aspects, a condition may refer to a patient's state of health or physical fitness or the state of health or physical fitness of an organ or anatomical part of a patient. In certain embodiments, a condition may refer to an illness, pain, discomfort, defect, disease, or deformity of a patient or of an organ or anatomical part of a patient. (Search “condition” on wordhippo.com. WordHippo, 2021. Web. Accessed 8 Dec. 2021. Modified.)

“Deflator” refers to a structure, device, component, member, system, assembly, and/or part configured deflate or cause deflation of a structure, container, system, device, or apparatus. Generally, a deflator is configured to release and/or remove a fluid such as a liquid or a gas from a closed or closable system such that the system has less fluid or fluids within the system experience a decrease in pressure within the system. An deflator can be a single component or structure or a plurality of components or structures or an assembly. The deflator can be mechanical, pneumatic, hydraulic, manual, chemical, thermal, pyrotechnic, electronic, or electromechanical.

“Inflated state” refers to a state or condition of a container in which the container holds a desired level of a fluid. In certain aspects, an inflated state may include a state in which a container deforms and may expand to manage a fluid and/or pressure of the fluid within the container.

“Inflation state” refers to a state or condition of inflation of a container, vessel, space, or volume. An inflation state is a measure of a degree and/or quality of inflation of a vessel at a given point in time. An inflation state can be expressed as a percentage with 0% being an uninflated state and 100% being a fully inflated state. Examples of inflation states include, but are not limited to, full inflation or fully inflated, under inflated, partially inflated, not inflated, no inflation, uninflated, over inflated, and the like. In certain embodiments, an over inflated state is a condition in which the vessel is inflated to a pressure beyond its designed capacity or inflated to a pressure level that containment of a fluid by the vessel fails and fluid is released.

As used herein, “coupling”, “coupling member”, or “coupler” refers to a mechanical device, apparatus, member, component, system, assembly, or structure, that is organized, configured, designed, arranged, or engineered to connect, or facilitate the connection of, two or more parts, objects, or structures. In certain embodiments, a coupling can connect adjacent parts or objects at their ends. In certain embodiments, a coupling can be used to connect two shafts together at their ends for the purpose of transmitting power. In other embodiments, a coupling can be used to join two pieces of rotating equipment while permitting some degree of misalignment or end movement or both. In certain embodiments, couplings may not allow disconnection of the two parts, such as shafts during operation. (Search “coupling” on Wikipedia.com Jul. 26, 2021. CC-BY-SA 3.0 Modified. Accessed Jul. 27, 2021.) A coupler may be flexible, semiflexible, pliable, elastic, or rigid. A coupler may join two structures either directly by connecting directly to one structure and/or directly to the other or indirectly by connecting indirectly (by way of one or more intermediary structures) to one structure, to the other structure, or to both structures.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. The phrase “fluid communication” refers to two features that are connected such that a fluid within one feature is able to pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Conventional earwax removal tools and/or techniques have been in place for some time. However, these tools and techniques have limitations and can often present several challenges, including the risk of injury to the ear canal or eardrum, difficulty in controlling insertion depth, and the inability to effectively remove earwax without causing discomfort or complications. These challenges can lead to patient dissatisfaction and may even result in adverse events or complications. However, the innovative features and advantages of the disclosed earwax removal systems, apparatuses, tools, and/or methods address and/or remediate these problems, offering a safer, more efficient, and patient-friendly solution.

One challenge with existing earwax removal tools is the lack of a reliable mechanism to prevent insertion of tools, or parts of a tool, too far into the ear canal, which can lead to injury or discomfort. The incorporation of a stop in the design of the present disclosure serves as a hard physiologic stop, leveraging the natural boundary of the ear canal and preventing over-insertion. This feature ensures that the tool can be used safely by patients without the risk of injuring the ear canal or eardrum.

Additionally, the reusable handle/body of a device according to the present disclosure combined with replaceable/disposable/interchangeable tips offers a cost-effective and hygienic solution. In one embodiment, the tip is reusable. In certain embodiments, the tips may be disposable. Patients can easily replace the disposable tips after each use, minimizing the risk of contamination and ensuring optimal hygiene standards. Of course, the same tip can be used for both ears of a particular patient or user or even a plurality of users. In certain embodiments, the same tip can be cleaned and/or sterilized between uses for a single user and/or between use on different users.

In certain embodiments, a handle/body of a device according to the present disclosure may be configured to enable a user to use and manipulate the device with one hand and/or with either a left hand or a right hand. These handling features enhance usability and convenience, allowing users to readily perform an earwax removal procedure and/or patients to self-administer earwax removal safely and effectively.

Another advantage of the proposed tool/apparatus is the inclusion of an effective, durable, and reliable driver and/or inflator mechanism, which can be implemented in a variety of embodiments, such as a piston, a syringe, or other suitable apparatus. The inflator enables controlled inflation of the balloon tip, preventing overinflation and ensuring that the balloon presses against the walls of the ear canal with the appropriate pressure for effective earwax extraction. In certain embodiments, the inflator includes a predefined volume of fluid and is configured together with the balloon to provide a pressure regulating mechanism to prevent overinflation. Additionally, the design of the balloon as an elastic and firm structure, combined with the use of water as the inflation fluid/medium, further enhances its effectiveness in removing earwax without causing discomfort to the patient.

Furthermore, the flexibility of the shaft and the ergonomic design of the handle provide ease of insertion and removal, enhancing patient comfort and minimizing the risk of injury during the earwax removal process. In certain embodiments, the inclusion of a controlled deflation mechanism and a reliable coupling between the balloon and the shaft further improves the safety and efficacy of the tool/apparatus, ensuring that the earwax extraction process is controlled and precise.

In summary, the innovative features and advantages of the proposed, presented, and presently disclosed system, apparatus, tool, device, and/or method for earwax removal address and/or remediates the limitations of existing tools effectively. By incorporating safety mechanisms, usability enhancements, and optimized design features, the present disclosure provides a safer, more efficient, and patient-friendly solution for earwax removal, ultimately improving patient satisfaction and reducing the risk of complications.

FIG. 1 is a block diagram illustrating one embodiment of a system 100 for removing cerumen from a subject or patient according to one embodiment. Throughout this disclosure reference may be made to a patient and/or a subject or both. Either the term “subject” or “patient” may be used, and the intent is that such use conveys the broadest interpretation of either term in relation to the context in which the term is used. The system 100 includes a driver 110, a fluid supply 120, a balloon 130, a handle 140, and a stop 150.

The driver 110 controls the flow of fluid into the balloon 130. In one embodiment, the driver 110 controls fluid flow into the balloon 130 and controls fluid flow out of the balloon 130. In certain embodiments, the driver 110 controls flow of fluid from the fluid supply 120 and into the balloon 130. Alternatively, or in addition, the driver 110 can also control the flow of fluid 122 between the balloon 130 and the fluid supply 120. The driver 110 may comprise a fluid coupling with the fluid supply 120 and the balloon 130. In one embodiment, the driver 110 is coupled to the balloon 130 by a fluid conduit that conveys the fluid into and/or out of the balloon 130.

In certain embodiments, the driver 110 is coupled to the balloon 130 such that activation of the driver 110 forces, or drives, a fluid 122 into the balloon 130. In one embodiment, the forcing of the fluid 122 into the balloon 130 inflates the balloon 130. In one embodiment, activation of the driver 110 inflates the balloon 130 with fluid 122. The fluid 122 used to inflate the balloon 130 may come from the fluid supply 120. Alternatively, or in addition, activation of the driver 110 may also cause, or force, fluid 122 to move out of the balloon 130. The action of fluid 122 moving out of the balloon 130 may deflate the balloon 130.

In one embodiment, the driver 110 causes fluid 122 to move from the fluid supply 120 and into the balloon 130 with sufficient pressure to inflate the balloon 130. Alternatively, or in addition, the driver 110 may also cause fluid 122 to move from the balloon 130 to the fluid supply 120 to effectively remove, release, or withdraw fluid 122 from the balloon 130. This action can deflate the balloon 130. In one embodiment, the driver 110 may simply release pressure on the fluid 122 in the balloon 130 to deflate the balloon 130. In another embodiment, the driver 110 may draw, pump, drive, and/or remove the fluid 122 from the balloon 130 to deflate the balloon 130. For example, in one embodiment, the driver 110 is configured such that deactivation of the driver 110 removes fluid 122 from the balloon 130. Alternatively, or in addition, the system 100 may be configured such that activation of another driver removes fluid 122 from the balloon 130. In certain embodiments, deactivation of the driver 110 enables pressure of fluid 122 in the balloon 130 to decrease and this enables the balloon 130 to at least partially deflate.

Those of skill in the art will appreciate that a variety of different apparatuses, devices, systems, and/or components may serve as the driver 110 or as part of the driver 110. In one embodiment, the driver 110 can be a simple valve with fluid 122 under pressure on one side of the valve such that opening the valve causes fluid 122 to flow into the balloon 130. In another embodiment, the driver 110 may be mechanical pump. In another embodiment, the driver 110 may be an electrical pump. In another embodiment, the driver 110 may be an electromechanical pump. In yet another embodiment, the driver 110 may be closed fluid system with a deformable side or wall such that a user applying pressure on an outside of the deformable wall forces fluid 122 into the balloon 130. In one embodiment, the driver 110 comprises a syringe, a pump, a compressor, an impeller, a turbine, a propeller, an agitator, and injector, or the like.

The driver 110 is designed to be easy to use and intuitive, with simple controls that allow the user to manage the flow rate and pressure of the fluid 122. The driver 110 can be used to change, manage, and/or control an inflation state for the balloon 130. In one embodiment, the driver 110 is used to increase inflation of the balloon 130, maintain an inflation state, decrease and inflation state, or deflate the balloon 130. The driver 110 is also designed to be durable and reliable, with high-quality components that can withstand the rigors of regular use.

The fluid supply 120 stores and delivers a fluid 122 that is used to inflate the balloon 130. In one embodiment, the fluid 122 is water. In another embodiment, the fluid 122 is an oil or oil-based mixture. In one embodiment, the fluid supply 120 is a container in fluid communication with the driver 110. In certain embodiments, the inside of the fluid supply 120 and its contents are pressurized such that opening a valve coupled to the fluid supply 120 releases fluid 122 within the fluid supply 120. In one embodiment, the fluid supply 120 is a closed system. In another embodiment, the fluid supply 120 is an open system such that pressure within the fluid supply 120 can be balanced with atmospheric pressure for the system 100. In an open system, the fluid supply 120 may include a fluid 122 that is under atmospheric pressure (i.e., pressure on fluid 122 within the fluid supply 120 is the same as the atmospheric pressure around the fluid supply 120.)

In one embodiment, the fluid supply 120 is a container that holds a single fluid 122 such as air or water. In another embodiment, the fluid supply 120 holds a mixture, combination, or solution of fluids 122 such as a mixture of air and water. In one embodiment, the fluid 122 may be a gas and/or a gas mixture, such as air, nitrogen, or the like.

In certain embodiments, the fluid supply 120 is configured to hold a sufficient volume of fluid 122 for use inflating and/or deflating the balloon 130. The fluid supply 120 may have a large capacity that allows for extended use without the need for frequent refills. Alternatively, or in addition, the fluid supply 120 may hold more than enough fluid 122 for inflating and/or deflating the balloon 130. In another embodiment, the fluid supply 120 is configured to be refillable such that more fluid 122 can be added, as needed. In certain embodiments, the driver 110 and/or a fluid conduit connecting the driver 110 and the balloon 130 may be used to refill or add more fluid 122 to the fluid supply 120. In one embodiment, the fluid supply 120 is designed to be safe and secure, with features that prevent spills and leaks.

The balloon 130 serves as an adaptable, reconfigurable, cerumen extraction device. The balloon 130 is configured to be inserted into an ear canal in a deflated state such that the deflated balloon 130 mitigates, or avoids, pushing or compacting cerumen in the ear canal deeper into the ear canal. In one embodiment, in a deflated state, the balloon 130 is configured to have a minimal cross-sectional diameter. In one embodiment, the cross-sectional diameter of the balloon 130 in a deflated state enables the balloon 130 to move past cerumen in the ear canal without disturbing the cerumen.

In addition, the balloon 130 is configured to change its shape (e.g., its cross-sectional diameter) once within the ear canal such that extraction of the balloon 130 pushes cerumen on the walls of the ear canal out of the ear canal towards a distal opening of the ear canal. In one embodiment, the balloon 130 increases its diameter by being inflated by a fluid 122. In one embodiment, the balloon 130 is configured to inflate to a shape having about a 5 mm diameter. In one embodiment, the balloon 130 is coupled to the fluid supply 120 by a fluid conduit that provides fluid communication between fluid 122 in the fluid supply 120 and at least one inside chamber of the balloon 130. In one embodiment, activation of the driver 110 forces fluid 122 into the balloon 130 and increases the balloon's 130 diameter. In one embodiment, activation of the driver 110 inflates the balloon 130.

In certain embodiments, the inflation force of the driver 110 is configured to inflate the balloon 130 without causing the balloon 130 to fail or break. In one embodiment, the driver 110 and/or quantity of fluid 122 in the fluid supply 120 is configured such that overinflation of the balloon 130 is not possible. Alternatively, or in addition, the fluid delivery mechanism, such as a shaft together with the driver 110 and/or quantity of fluid 122 and/or compressibility of the fluid 122 in the fluid supply, in certain embodiments, is configured to prevent and/or mitigate overinflation of the balloon 130.

In certain embodiments, the balloon 130 is coupled to the fluid supply 120 by a shaft. The shaft is one example of a fluid conduit. In one embodiment, the shaft is a slender, cylindrical structure made of a lightweight and biocompatible material such as nylon, plastic, or paper. The shaft serves to hold the balloon 130 and convey fluid between the balloon 130 and the driver 110. The shaft is, and is designed to be, rigid enough to maintain its shape and structural integrity but flexible enough to allow for gentle and precise movements. In one embodiment, the driver 110 is coupled to the shaft. In another embodiment, the driver 110 is couplable to the shaft. In yet another embodiment, the driver 110 is coupled to an internal opening of the shaft, the internal opening extending from one end of the shaft to the other end of the shaft.

In one embodiment, the cross-sectional diameter of the shaft and the cross-sectional diameter balloon 130 (in a deflated state) are each sized to fit within an auditory canal of a patient. In one embodiment, the shaft and balloon 130 may be coaxial, and the cross-sectional diameter of the shaft and balloon 130 together may be smaller than a diameter of the auditory canal of the patient. In certain embodiments, the cross-sectional diameter of the shaft and balloon 130 together may be smaller than a diameter of the auditory canal of the patient and a height of any build-up of cerumen on one or more walls of the auditory canal of the patient.

In one embodiment, the balloon 130 is configured to have a minimal cross-sectional diameter when deflated and a suitable cross-sectional diameter when inflated. In one embodiment, the suitable cross-sectional diameter may be just greater than a cross-sectional diameter of the ear canal of the patient. The balloon 130 includes at least one inlet port. The balloon 130 can have a variety of shapes in both an inflated and/or deflated state. In one embodiment, the balloon 130 has an elongated narrow shape when deflated. In one embodiment, the uninflated balloon 130 has a cylindrical shape and may have a length of about 9.95 mm and a diameter of about 3.4 mm. This shape may facilitate insertion into an ear canal. In one embodiment, the inflated balloon 130 has a spherical shape and may have a diameter of about 10 mm.

In certain embodiments, the balloon 130 includes a single inlet and a shaft extends into the inside of the balloon 130. The portion of the shaft extending into the balloon 130 may serve as a structural support to hold and move the balloon 130 into place as the balloon 130 is inserted and/or extracted from an ear canal.

In one embodiment, the balloon 130 has an elongated thin shape when deflated. The balloon 130 may have a plurality of shapes when inflated. For example, the inflated balloon 130 may have a sphere shape, a disc shape, and the like. Here are a few possible examples for shapes for the balloon 130 according to the present disclosure:
Spherical: A round balloon shape that conforms to the shape of the ear canal.
Oval: An elongated balloon shape that provides more surface area for contact with cerumen.
Teardrop: A shape resembling a teardrop, with a tapered end for easier insertion into the ear canal.
Conical: A cone-shaped balloon that gradually tapers toward the tip for efficient cerumen removal.
Bulbous: A balloon shape that is bulbous or rounded at the end to effectively trap and extract cerumen.
Cylindrical: A cylindrical balloon shape that fits snugly within the ear canal for thorough cleaning.
Irregular: A balloon shape with irregular contours designed to reach and remove cerumen from difficult-to-reach areas.
Tapered: A balloon shape that tapers from a wider base to a narrower tip, facilitating insertion and extraction.
Flared: A balloon shape that flares outward at the end to create a seal and prevent cerumen from escaping.
Spiral: A balloon shape with a spiral pattern or helical design for enhanced cerumen removal through rotational movement. (© ChatGPT 3.5 Version, Modified, accessed chat.openai.com/chat around Apr. 19, 2024).

The balloon 130 may be made from a variety of materials. The materials may be used or chosen to provide factors such as biocompatibility, mechanical properties, flexibility, sterilizability, and compatibility with the system's 100 intended use. These materials may include natural rubber latex (NRL), synthetic polymeric materials such as polyethylene, polyurethane, and silicone, thermoplastic elastomers (TPE), nylon, and composite materials. NRL balloons, derived from the sap of the rubber tree, offer high elasticity and flexibility but may pose allergic risks. Synthetic polymeric materials, including polyethylene, polyurethane, and silicone, afford versatility and customizable properties suited for various medical applications. Thermoplastic elastomers (TPEs) combine thermoplastic and elastomeric characteristics, offering excellent elasticity, flexibility, and biocompatibility. Nylon balloons provide strength, durability, and tear resistance, making them suitable for use as the balloon 130.

Composite materials offer tailored mechanical and chemical properties, while coatings, such as hydrophilic coatings, can enhance lubricity and reduce friction during balloon inflation and deflation. In certain embodiments, the balloon 130 is configured to inflate to a volume defined at least in part by the size of the inside of the balloon 130 and/or by a degree of flexibility and/or elasticity of the balloon 130. Alternatively, or in addition, the balloon 130 is configured to stretch (e.g., is elastic) when inflated to take on a volume larger than the size of the inside of the balloon 130 in an uninflated state.

In one embodiment, the balloon 130 is made from high-quality materials that are designed to be strong and durable, with a smooth surface that allows for easy insertion and removal. In another embodiment, the balloon 130 includes a textured surface. The textured surface may facilitate engagement with cerumen and collection of cerumen as the balloon 130 is used. Features of the textured surface may be of a variety of shapes, sizes, and/or configurations. In one embodiment, the features of the textured surface may include ridges, waves, dimples, protrusions, bulges, elevations, a combination of these, or the like. The balloon 130 may also include features to facilitate easy cleaning and sterilization, and that minimizes a risk of contamination.

The handle 140 serves to facilitate use and manipulation of the system 100. In one embodiment, the handle 140 may be connected to, or may couple to, a body or housing of the system 100. The handle 140 is coupled, or connected, to the balloon 130 such that a user grasping the handle 140 can readily insert the balloon 130 into an ear canal of a patient. The handle 140 also enables a user to readily remove the balloon 130 from the ear canal when the balloon 130 is inflated. In certain embodiments, the handle 140 may enable or facilitate a user readily inflating the balloon 130 as the balloon 130 is within the ear canal.

In certain embodiments, the handle 140 is a component that the user holds onto while using the system 100. The handle 140 may be designed to be comfortable and ergonomic, with a shape that fits comfortably in the hand and reduces fatigue during extended use. The handle 140 is also designed to be durable and reliable, with high-quality materials that can withstand the rigors of regular use.

In one embodiment, the handle 140 may include a grip 142. The grip 142 facilitates engagement with the hand of a user and facilitates manipulation of the system 100. In certain embodiments, the grip 142 is configured to receive at least four fingers of a user holding the handle 140. In addition, in certain embodiments, the grip 142 may be configured to engage with a palm of the user. In one embodiment, a thumb of the user may not engage with the handle 140. In another embodiment, the grip 142 may include a thumb grip, positioned, configured, and/or oriented to engage with a thumb of a user as the user uses the system 100.

The stop 150 is a safety feature that prevents over-insertion of the balloon 130 within the ear canal. Over-insertion is desirable to prevent damage, pain, or discomfort to the part of the ear on a proximal end of the ear canal, such as the ear drum. Advantageously, the stop 150 limits an insertion distance of the balloon 130 into an ear canal of a subject.

in one embodiment, the stop 150 is coupled to the shaft. The shaft may also be coupled to the balloon 130. The stop 150 may be coupled to the shaft such that the stop 150 prevents contact between one or both of a distal end of the shaft and the balloon 130 and a tympanic membrane of the patient, when the shaft is inserted into the ear canal. In another embodiment, the stop 150 mitigates or limits contact between a distal end of the shaft and the tympanic membrane.

The stop 150 is designed to contact a tragus and/or anti-tragus of the ear of the patient such that the balloon 130 connected to the handle 140 cannot be inserted further into the ear canal. In one embodiment, the stop 150 is positioned and connected to the balloon 130, such as by way of the shaft, such that once the stop 150 contacts the tragus and/or anti-tragus or another part of the ear near the distal opening of the ear canal a user can not push the balloon 130, and/or a shaft coupled to the balloon 130, deeper into the ear canal. The stop 150 is simple, effective, reliable, and accurate. In one embodiment, the stop 150 is a separate component. In another embodiment, the stop 150 is integrated into another component or part of the system 100.

In one embodiment, the system 100 is configured such that the patient and the user are, or can be, the same person. Thus, a user can remove cerumen from their own ear using the system 100. In such embodiments, the system 100 may be adapted to the size and/or age of the user. For example, if the user is a child or adolescent, the shaft may be shorter and/or the stop 150 may be larger. In certain embodiments, the handle 140 is configured for ambidextrous use by the user, such that a user can remove cerumen from each of their ears using either hand. In another embodiment, the user and the patient may be two distinct persons (e.g., a medical professional and the user).

The system 100 can be used with a variety of different kinds, types, and/or configurations of drivers 110.

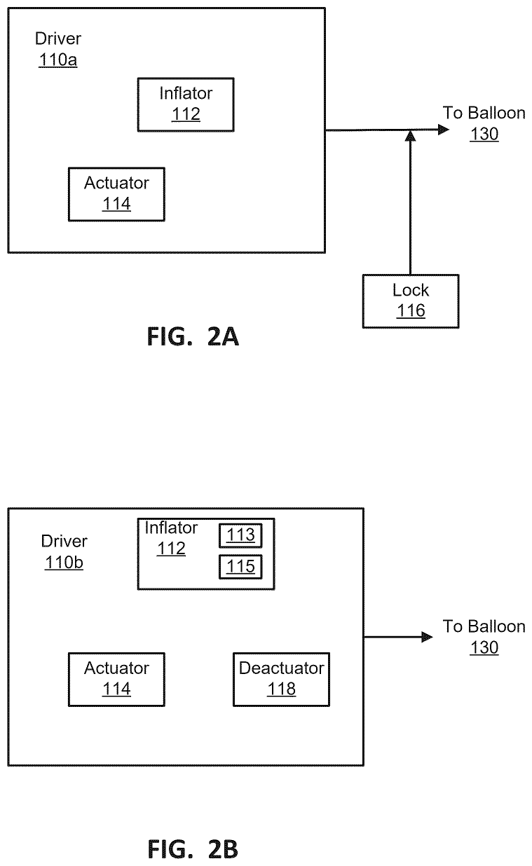

FIG. 2A is a block diagram illustrating one embodiment of a driver 110a for use in removing cerumen from a patient according to one embodiment. In certain embodiments, the driver 110 includes an inflator 112 and an actuator 114.

The inflator 112 serves to inflate the balloon 130. In one embodiment, the inflator 112 inflates the balloon 130 by increasing a pressure of fluid 122 within the balloon 130 by forcing more fluid into the balloon 130. This type of pressure is fluid pressure or hydraulic pressure. In one embodiment, the inflator 112 is in fluid communication with a passage of a shaft such that as the inflator 112 forces fluid through a passage of the shaft, the balloon 130 is inflated. In one embodiment, the inflator 112 is configured to inflate the balloon 130 by forcing a fluid through the passage.

In certain embodiments, not only can the inflator 112 increase the pressure of fluid 122 in the balloon 130 but the inflator 112 may also be configured to decrease a pressure of fluid 122 in the balloon 130. In one embodiment, the inflator 112 may decrease the pressure by reversing an operation done that increased the pressure. In another embodiment, the inflator 112 may decrease the pressure by releasing pressure from the balloon 130. In a closed fluid system that includes the balloon 130, pressure may be decreased by, for example, opening a valve.

In certain embodiments, the inflator 112 may be a mechanical device and/or component. In another embodiment, the inflator 112 may be a motorized pump or device. In still another embodiment, the inflator 112 can be a structure that can be deformed to change a volume of a closed fluid system that includes the inside of the balloon 130.

In another embodiment, the inflator 112 may inflate the balloon 130 by decreasing a volume of a closed system that includes the inside of the balloon 130. For example, the inflator 112 may mechanically compress a part, or component, of a closed fluid system in fluid communication with the balloon 130 such that the compression decreases the volume of the system which then forces fluid 122 into the balloon 130.

In the illustrated embodiment, the inflator 112 may be controlled by the actuator 114. The actuator 114 serves to manage, control, and/or operate the inflator 112. Where an inflator 112 is implemented as an electric pump, an actuator 114 may be a switch that activates the pump. Where the inflator 112 is implemented as a mechanical pump, the actuator 114 may be a piston, a lever, a bellows, a rod, or the like that may be manually driven to activate the mechanical pump.

In certain embodiments, an actuator 114 may have a single function of activating the inflator 112 to increase the pressure of (or on) fluid 122 within the balloon 130. In another embodiment, the actuator 114 may have two or more functions. One function may be to activate the inflator 112 to increase the pressure on fluid 122 within the balloon 130. A second function of the same actuator 114 may be to activate the inflator 112 to decrease the pressure on fluid 122 within the balloon 130. For example, the actuator 114 may activate a motor inflator 112 or pump inflator 112 to operate in reverse. In such an example, the pump inflator 112 may suck or extract fluid 122 from the balloon 130 rather than drive or pump fluid 122 into the balloon 130. In one embodiment, the actuator 114 may be operated or controlled by a user of the system 100.

In certain embodiments, the system 100 may also include a lock 116. The lock 116 may serve to maintain an inflation state for fluid 122 within the balloon 130. For example, if the balloon 130 is fully inflated, the lock 116 may prevent deflation and/or further inflation of the balloon 130. Alternatively, or in addition, if the balloon 130 is deflated, or at any inflation state between deflated and inflated, the lock 116 may prevent or mitigate inflation or further deflation of the balloon 130. In certain embodiments, the lock 116 may provide and/or facilitate a controlled deflation of the balloon 130.

Of course, those of skill in the art will appreciate that the lock 116 may be implemented in a variety of ways using a variety of structures and/or devices. In one embodiment, the lock 116 is a valve that can be closed or partially closed to prevent fluid 122 from entering or exiting the balloon 130. The valve may be a mechanical valve. In another embodiment, the lock 116 may be a switch or control on the actuator 114 and/or inflator 112 that prevents activation and/or deactivation of the actuator 114 and/or inflator 112 which would change the pressure of fluid 122 in the balloon 130.

FIG. 2B is a block diagram illustrating one embodiment of a driver 110b for use in removing cerumen from a patient according to one embodiment. The driver 110b may have many structures, features, and functions, operations, and/or configuration similar or identical to those of the driver 110a described in relation to FIG. 2A, like parts are identified with the same or similar reference numerals. Accordingly, the driver 110b may include an inflator 112 and an actuator 114. In addition, the driver 110b may also include a deactuator 118.

The actuator 114 of the driver 110b may differ from the actuator 114 of the driver 110a in that the actuator 114 of the driver 110b may perform a single function of activating the inflator 112 to increase the pressure on fluid 122 within the balloon 130, rather than activating the inflator 112 both to increase the pressure and to decrease the pressure. In the driver 110b, the deactuator 118 may provide the feature of activating the inflator 112 to decrease the pressure on fluid 122 within the balloon 130.

In certain embodiments, the actuator 114 is configured to activate the inflator 112 to inflate the balloon 130. In one embodiment, the inflator 112 includes a syringe. The syringe may include a barrel that includes fluid. The barrel may include a distal end with a distal opening and a proximal end with a proximal opening. The distal opening may be configured to interface with a passage of a shaft to enable fluid flow between the barrel and the passage in response to operation of the actuator 114. The actuator 114 in one embodiment, may include a plunger that extends from a proximal opening of the barrel and extends from a cerumen removal device.

As with the actuator 114, the deactuator 118 may be implemented using a variety of different structures, parts, and/or components. Examples include a switch, a lever, a knob, a valve, a controller, a control lever, a button, a catch, a latch, or the like. In one embodiment, the deactuator 118 is a mechanical structure that operates to counter act the action of the inflator 112.

For example, suppose the inflator 112 is a pump or a bellows, or a syringe and the inflator 112 is in fluid communication with fluid 122 in the interior of the balloon 130. In such an example, the deactuator 118 may be a biasing member that is biased when the inflator 112 is activated to inflate the balloon 130 and when the inflator 112 stops or is in a stable condition, the biasing member has stored a biasing force that the biasing member then imparts on the inflator 112 to reverse the inflation operation such that the balloon 130 deflates. In one embodiment, the deactuator 118 implemented as a biasing member is a coil spring. Of course, other kinds and/or types of springs can be used for the deactuator 118.

In one embodiment, the inflator 112 includes a chamber 113 and a driver 115. The chamber 113 includes a fluid 122 and an outlet that provides fluid communication between the fluid in the chamber 113 and the balloon 130. In one embodiment, the outlet provides fluid communication between the fluid 122 in the chamber 113 and a shaft that is coupled to the balloon 130. In certain embodiments, the shaft may include a longitudinal passage from one end to the other. In one embodiment, the driver 115 can be configured to pressurize the chamber 113 such that fluid 122 in the chamber 113 flows through the passage and into the balloon 130.

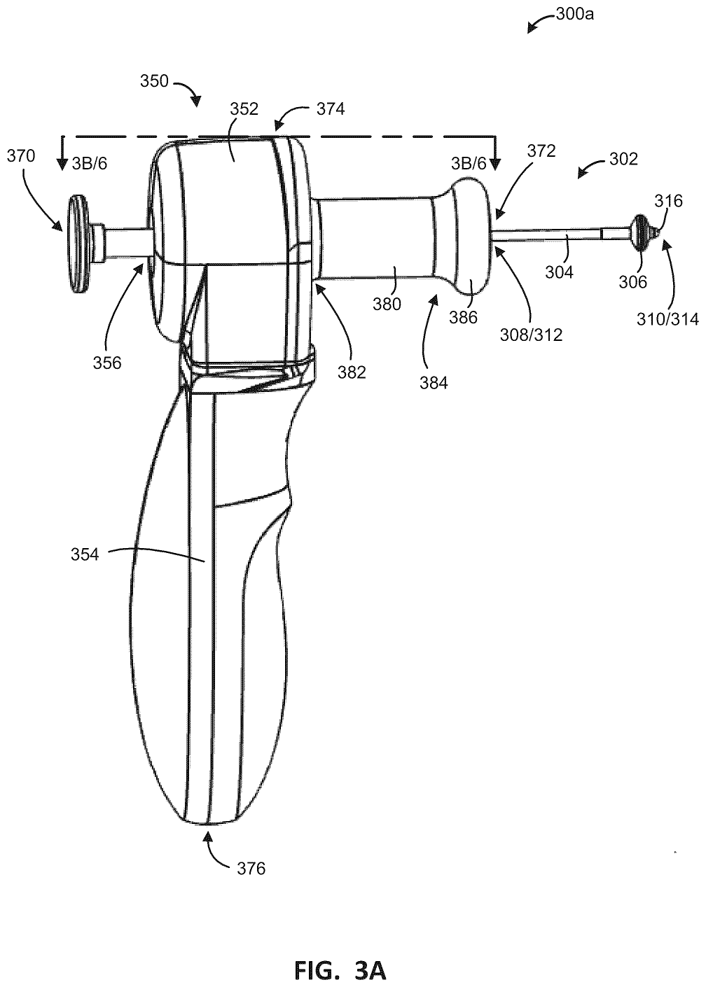

FIG. 3A is a perspective view of one embodiment of an apparatus 300a for removing cerumen from a patient according to one embodiment. The apparatus 300a includes a tip 302 and a body 350. The tip 302 includes a shaft 304 and a balloon 306. The body 350 includes a head 352, a handle 354, and an inflator 356.

The tip 302 is configured to be inserted into an ear canal of a patient. The tip 302 is the part a user inserts into the ear canal to remove cerumen. In certain embodiments, the tip 302 is fixed to the body 350. In another embodiment, the tip 302 is removable such that one tip 302 can be replaced with another tip 302. Alternatively, or in addition, a variety of different kinds or types of tips 302 can be used with a body 350.

The tip 302 includes a proximal end 308 and a distal end 310. In one embodiment, the proximal end 308 is connected to the body 350. In another embodiment, the proximal end 308 includes a coupling configured to couple the tip 302 to the body 350 for use by a user. In one embodiment, the coupling provides a fluid connection between the balloon 306 and an inflator. Advantageously, the coupling is one that can be readily connected and/or disconnected by an untrained user such that the tip 302 can be readily removed, exchanged, attached, or the like. In one embodiment, the coupling is a friction fit between a proximal end 308 of the tip 302 and a distal end of the body 350. Of course, other types of couplings may be used. For example, in one embodiment, a luer lock (either a slip luer lock or a threaded luer lock) may be used as the coupling between the tip 302 and the body 350.

In certain embodiments, a tip 302 may be replaceable and/or exchangeable. A different tip 302 used in place of an original tip 302 may be substantially the same or similar to the tip 302 in the illustrated embodiment. In this manner, a user may exchange a used tip 302 (one that includes extracted cerumen) with a clean, fresh, sanitized, new tip 302 for use on the same ear canal or another ear canal of the same patient or of another patient.

In another embodiment, tip 302 is one example of a plurality of kinds and types of tips 302 that can be used with the apparatus 300a. These other tips 302 (alternative tips) may be used for a variety of purposes. Some alternative tips may include a balloon 306 and others may have a different configuration and/or not include a balloon 306. One example of an alternative tip may include a tip with a shaft 304 and a medicine, drug, salve, crème, ointment or the like within a fluid supply 120. In such an embodiment, operation of the inflator 356 may force fluid 122 in the fluid supply 120 into an internal proximal portion of the ear canal of a patient.

In another example, the alternative tip may not be in fluid communication with the fluid supply 120. Instead, the alternative tip may include a shaft 304 and may include a pad or absorbent structure or another structure near a distal end of the shaft 304. A pad or absorbent structure on the shaft 304 may be used to absorb a fluid in the ear canal, (e.g., blood, or a fluids) from an infection in the ear. Alternatively, or in addition, the pad or absorbent structure may be used to deposit a fluid or substance such as a medication, a drug, and antibiotic or the like, within the ear canal. In another embodiment, a pad or other structure at or near the distal end of the shaft 304 may be used to contact and/or scratch an inside wall of the ear canal of a patient. This kind of tip can be useful to relieve discomfort from an itch.

The shaft 304 supports and connects the balloon 306 to the body 350. In addition, the shaft 304 provides fluid communication between the interior of the balloon 306 and an inflator of the body 350. The shaft 304 also includes a proximal end 312 and a distal end 314. In the illustrated embodiment, the proximal end 312 of the shaft 304 corresponds to the proximal end 308 of the tip 302 and the distal end 314 of the shaft 304 corresponds to the distal end 310 of the tip 302.

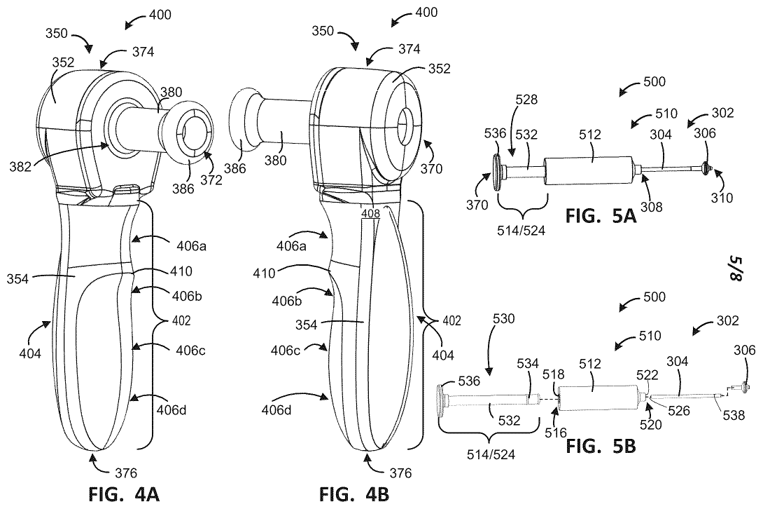

In one embodiment, the shaft 304 includes a passage 526 (See FIG. 5B) that extends from the proximal end 312 to the balloon 306. The shaft 304 may also include an opening 538 (See FIG. 5B) along the shaft 304 that connects the passage to the interior of the balloon 306. The passage enables fluid communication between the proximal end 312 of the shaft 304 and the balloon 306. In certain embodiments, the passage is coaxial with a longitudinal axis of the shaft 304.

The shaft 304 may be the same, or similar to, the shaft described herein in relation to the system 100. Thus, in certain embodiments, the shaft is a slender, cylindrical structure made of a lightweight and biocompatible material such as nylon, plastic, or paper. In one embodiment, the shaft 304 is a flexible tube with an inner diameter of about 1.52 mm, an outer diameter of about 1.75 mm and a length of about 15 mm.

The shaft serves to hold the balloon 306 and convey fluid between the balloon 306 and a driver 110 or inflator. The shaft 304 is, and is designed to be, rigid enough to maintain its shape and structural integrity but flexible enough to allow for gentle and precise movements. In one embodiment, the shaft 304 is implemented using tubing of between about 0.030″-0.080″ (about 1.5 mm to 2.5 mm) in diameter. In one embodiment, a driver 110 or inflator is couplable to the passage of the shaft 304.

The balloon 306 may be the same, or similar to, the balloon 130 described herein in relation to the system 100. In certain embodiments, the balloon 306 may be one example implementation of a plurality of possible embodiments of the balloon 130. Thus, the balloon 306 may include one or more or all of the aspects, features, and/or attributes of the balloon 130. The balloon 306 is in fluid communication with a passage of the shaft 304.

In the illustrated embodiment, the balloon 306 is secured and/or positioned to the shaft 304 near a distal end 314 of the shaft 304. Alternatively, or in addition, the balloon 306 may be secured and/or positioned to the shaft 304 at a distal end 314 of the shaft 304. In another embodiment, the balloon 306 may be secured and/or positioned to the shaft 304 at any position along a longitudinal length of the shaft 304. Positioning the balloon 306 near the distal end 314 can be advantageous because when the balloon 306 is positioned in the ear canal and inflated, the more distal the balloon 306 is along the shaft 304 the greater the likelihood and/or the quantity of cerumen the balloon 306 will contact and push out of the ear canal when the tip 302 is removed retrograde from the ear canal.

In one embodiment, the balloon 306 is secured to the shaft 304 by an adhesive. The balloon 306 may be connected to the shaft 304 by an ultraviolet (UV) activated adhesive. Alternatively, or in addition, the balloon 306 may be heat treated to shrink the balloon 306 to strengthen a bond between the balloon 306 and the shaft 304.

In the illustrated embodiment, the balloon 306 may be positioned near the distal end 314 but not at the distal end 314. The tip 302 may further include a soft cap 316 secured to a distal end 314 of the shaft 304. The cap 316 may further protect against scratching or damage to the ear canal and/or tympanic membrane when the apparatus 300a is used.

In the illustrated embodiment, the body 350 includes a head 352, a handle 354, and an inflator 356. The body 350 has a proximal end 370, a distal end 372, a superior end 374, and an inferior end 376. In one embodiment, the body 350 may be about 120 mm between the superior end 374 and the inferior end 376.

The distal end 372 of the body 350 is configured to couple and/or is couplable to the shaft 304. In one embodiment, the distal end 372 is couplable to a proximal end 312 of the shaft 304 of the tip 302.

The head 352 serves to house the inflator 356. In the illustrated embodiment, the head 352 connects and/or is connectable to the tip 302. Specifically, in the illustrated embodiment, the head 352 is configured to couple to the shaft 304 of the tip 302. More specifically, the head 352 is configured to couple to the proximal end 312 of the shaft 304.

The head 352 may be made from a durable lightweight material such as plastic. Of course, other materials may be used as well. In one embodiment, the head 352 is a hollow structure that contains other parts of the apparatus 300a (e.g., the inflator 356 and/or a fluid supply 120). The head 352 may be just large enough to house the internal components and small enough to be lightweight and facilitate use and handling of the apparatus 300a by a user.

The head 352 is coupled to a handle 354. In the illustrated embodiment, the handle 354 is coupled or connected or connectable to the head 352 between the head 352 and the inferior end 376 of the body 350. The handle 354 provides an ergonomic structure that facilitates engagement and/or handling of the apparatus 300a by a user.

The design, configuration, features, and/or aspects of the handle 354 enable and facilitate use of the apparatus 300a by a user such as a medical technician, nurse, doctor or the like. In addition, the handle 354 of the present disclosure is configured such that the same apparatus 300a can be readily used by the patient to perform cerumen extraction from their own ears.

The ability to self-administer an earwax extraction procedure initially may seem straightforward. However, in order to perform the procedure, the patient will be using the apparatus 300a on a part of their own body that they cannot readily observe during the procedure. The apparatus 300a is designed to be used by inserting the tip 302 into the ear canal, specifically in one of the patient's own ears. Patients may use a mirror to assist in the procedure, but must be aware of the reflection effect and manipulate the apparatus in the opposite direction.

Thus, the process of using this apparatus 300a requires a certain level of coordination and dexterity on the part of the patient. In order to use the apparatus 300a on themselves, the patient must manipulate it in a specific way, which can be difficult due to the reflection effect caused by looking into a mirror. While a patient may choose to use a mirror to observe themselves during the procedure, this requires them to manipulate the apparatus in opposite directions to what is observed in the mirror.

Advantageously, the handle 354 is configured to facilitate use of the apparatus 300a by both the patient themselves and a user performing a cerumen extraction procedure on the patient. The handle 354 may be the same, or similar to, the handle 140 described herein in relation to the system 100. In certain embodiments, the handle 354 may be one example implementation of a plurality of possible embodiments of the handle 140. Thus, the handle 354 may include one or more or all of the aspects, features, and/or attributes of the handle 140.

The head 352 may enclose all or a portion of an inflator 356. The inflator 356 inflates the balloon 306 using fluid 122. The inflator 356 may be the same, or similar to, the inflator 112 described herein in relation to the system 100. In certain embodiments, the inflator 356 may be one example implementation of a plurality of possible embodiments of the inflator 112. Thus, the inflator 356 may include one or more or all of the aspects, features, and/or attributes of the inflator 112.

In the illustrated embodiment, the inflator 356 includes a portion that extends out of the head 352 towards the proximal end 370 of the body 350. The inflator 356 is coupled to the shaft 304 such that fluid 122 from the inflator 356 reaches the balloon 306. The inflator 356 is configured to inflate the balloon 306 by forcing a fluid 122 through the passage and into the balloon 306.

In certain embodiments, such as the illustrated embodiment, the body 350 includes a neck 380 having a proximal end 382 and a distal end 384. The neck 380 is coupled to the head 352 at the proximal end 382. The neck 380 may connect and/or support the inflator 356 and/or the shaft 304 of the tip 302.

In one embodiment, the neck 380 may be permanently connected to the head 352. Alternatively, or in addition, the neck 380 may couple to the head 352 by way of a coupler. In this manner, the tip 302 and neck 380 can be changed as needed. Alternatively, or in addition, the neck 380 may remain connected to the head 352 and the tip 302 may be changed, in certain embodiments. The neck 380 may include a fluid conduit that connects to the inflator 356 and a passage of the shaft 304. The neck 380 may be made of similar material as the head 352 and may include a smooth external surface. In one embodiment, the neck 380 has a circular cross section.

In one embodiment, the apparatus 300a includes a stop 386. The stop 386 may be the same, or similar to, the stop 150 described herein in relation to the system 100. In certain embodiments, the stop 386 may be one example implementation of a plurality of possible embodiments of the stop 150. Thus, the stop 386 may include one or more or all of the aspects, features, and/or attributes of the stop 150.

In one embodiment, the stop 386 is coupled to the distal end 384 of the neck 380. The stop 386 is configured to limit, stop, prevent, and/or mitigate an insertion distance of the shaft 304 into the ear canal of a patient. The stop 386 prevents over insertion of the tip 302 when the apparatus 300a is in use. The stop 386 is coupled to the body 350 such that the stop 386 prevents contact of the balloon 306 or the shaft 304 with a tympanic membrane of the subject, user, or patient.

The stop 386 may connect or couple to the distal end 384 of the neck 380 in a variety of ways. In one embodiment, the stop 386 is a silicone structure in the shape of a ring that engages with a rim or lip or edge of the distal end 384 of the neck 380. Alternatively, or in addition, the stop 386 may include threads and the distal end 384 may include corresponding threads such that the stop 386 can be screwed onto the distal end 384. In the illustrated embodiment, the stop 386 includes a curved and/or rounded external surface configured such that contact between the stop 386 and the ear or parts of the ear of a patient will not cause harm or damage.

Since the tip 302 is configured to be inserted into the ear canal of a patient atraumatically, the present disclosure includes one or more features to ensure that use of the apparatus 300a does not injure a patient or cause damage to sensitive parts of the ear such as the tympanic membrane (ear drum). The first feature is the length of the shaft 304. In one embodiment, a length of the shaft 304 from proximal end 312 to distal end 314 is between about 10 mm to about 28 mm. Another feature that mitigates injury to the patient is the use of a stop 386. The stop 386 prevents a user from inserting the tip 302 far enough to contact the tympanic membrane. Another feature that mitigates injury to the patient is the use of an option cap 316 on the distal end 314 of the shaft 304.

FIG. 3B a cross-section view illustrating a view along lines 3B/6-3B/6 of FIG. 3A, according to one embodiment of an apparatus 300b for removing cerumen from a subject/patient. The apparatus 300b may have many structures, features, and functions, operations, and/or configurations similar or identical to those of the apparatus 300a described in relation to FIG. 3A, like parts are identified with the same or similar reference numerals. Accordingly, the apparatus 300b may include a tip 302 and a body 350. The tip 302 includes a shaft 304 and a balloon 306. The body 350 includes a head 352, a handle 354, and an inflator 356.

As illustrated and described in relation to FIG. 3A, the tip 302 may include a shaft 304 and a balloon 306. The shaft 304 includes a passage 305 that extends from a proximal end 312 of the shaft 304. In one embodiment, the passage 305 extends from the proximal end 312 to an opposite end of the shaft 304 (i.e., the distal end 310). In another embodiment, the passage 305 extends from the proximal end 312 to point between the proximal end 312 and the distal end 310 of the shaft 304.

The balloon 306 is coupled to the shaft 304 at a position along, at, or near an end of the shaft 304. Said another way, the balloon 306 is coupled to the shaft 304 away from the proximal end 312. Furthermore, the balloon 306 is coupled to the shaft 304 such that the balloon 306 is in fluid communication with the passage 305. In one embodiment, an opening of the balloon 306 may be sealed around an external surface of the shaft 304.

The balloon 306 can have a variety of shapes, sizes, and/or configurations. In one embodiment, the balloon 306 and has a balloon cross-section diameter that is smaller than a canal cross-section diameter of an auditory canal of the subject. The smaller balloon cross-section diameter may be the state of the balloon 306 before the balloon 306 is inflated. Said another way, the balloon cross-section diameter may be smaller than a canal cross-section diameter of an auditory canal of the subject when the balloon 306 is at least partially deflated. This feature of the balloon 306 facilitates insertion and use of the balloon 306 into the auditory canal and mitigates a risk of the balloon 306 pushing cerumen deeper into the auditory canal.

The body 350 includes a head 352, a handle 354, and an inflator 356. The head 352 is configured to couple to the shaft 304 of the tip 302. The handle 354 is coupled to the head 352. The inflator 356 is in fluid communication with the shaft 304 and configured to inflate the balloon 306 by forcing fluid through the passage 305. The head 352 and handle 354 cooperate to facilitate placement of the tip 302 within an ear of a subject/patient. The handle 354 is ergonomic.

In the illustrated embodiment, the proximal end 312 of the shaft 304 is also the proximal end 308 of the tip 302. Advantageously, the proximal end 308/312 is configured to couple the tip 302 to the body 350 and/or, a component or part secured within the body 350, by way of a coupling 315 or coupler. In one embodiment, the coupling 315 may provide a permanent connection between the tip 302 and the body 350 or a component or part secured within the body 350. In another embodiment, the coupling 315 may provide a temporary connection between the tip 302 and the body 350 or a component or part secured within the body 350. In yet another embodiment, the tip 302 may be connected to the body 350 or a component or part secured within the body 350 without a coupling.

In embodiments in which the coupling 315 provides a temporary connection between the tip 302 and the body 350 or a component or part secured within the body 350, the coupling 315 may facilitate a replaceable feature of the tip 302. Alternatively, or in addition, the coupling 315 may provide a temporary coupling between the tip 302 and the body 350 or a component or part secured within the body 350 such that the tip 302 is interchangeable.

A replaceable and/or interchangeable tip 302 may enable use of the apparatus 300b with a variety of different tips 302. The different tips 302 may have different sizes, different shaft lengths, different size balloons 306, different numbers of balloons 306, different features for the tip 302, and the like. For example, the apparatus 300b may support adding a tip that includes a shaft with a textured surface such that the shaft can be used by a user to scratch inside walls of an ear canal to relieve itching. In another example, the apparatus 300b may support adding a tip that includes a shaft having a medicine, drug, salve, crème, ointment or the like one a surface of the shaft such that inserting the shaft deposits the a medicine, drug, salve, crème, ointment or the like inside and/or on the walls of an ear canal to remediate a condition of the patient/subject.

Those of skill in the art will appreciate that a variety of different kinds and/or types of couplings may be used for the coupling 315 to provide either a permanent coupling or a temporary coupling depending on the intended application. In some embodiments, threaded couplings may be employed, such as Luer lock couplings, which provide a secure, leak-proof connection, or internal/external threaded couplings, which use a male/female threading system to form a mechanically robust attachment.

Alternatively, bayonet couplings may be used to enable rapid quarter-turn engagement. In other embodiments, friction-fit and push-to-connect couplings may be utilized, including Luer slip couplings, which provide a quick, removable connection, or barbed couplings, wherein barbs on the shaft create an interference fit with the body 350 or a component or part secured within the body 350.

Additional examples include O-ring compression fittings, which rely on an elastomeric seal for enhanced fluid-tight engagement, and tapered press-fit couplings, which utilize conical mating surfaces to create a secure interface. For embodiments requiring rapid assembly and disassembly, quick-connect couplings may be used, such as snap-fit couplings, which engage via interlocking tabs, push-button quick-release couplings, which enable one-touch detachment, and magnetic couplings, which utilize magnetic attraction to maintain connectivity while allowing for separation when necessary.

In embodiments where permanent bonding is desired, adhesive-based couplings may be employed, such as epoxy-bonded couplings, where a biocompatible adhesive secures the connection, UV-cured adhesive couplings, which rapidly set upon UV light exposure, and solvent-welded couplings, wherein plastic components are chemically fused together. Additionally, mechanical locking couplings may be used, including set screw couplings, which secure the shaft via a lateral screw, clamp ring couplings, which use a circular clamp to apply radial pressure, and split-sleeve couplings, which rely on an adjustable sleeve to maintain engagement.

In specialized embodiments, medical-grade couplings may be implemented, such as rotary seal couplings, which permit rotational movement while maintaining fluid integrity, valved couplings, which prevent fluid leakage upon disconnection, and Swagelok compression couplings, which utilize ferrules to ensure high-pressure compatibility. Each of these coupling mechanisms offers distinct advantages depending on factors such as fluid pressure, rotational requirements, sterility, and ease of assembly, and may be selected accordingly to optimize performance within a given embodiment of the apparatus 300b.

FIG. 3B illustrates an apparatus 300b that includes a body 350 having a head 352 and handle 354 and the head 352 includes a neck 380 having a proximal end 382 and a distal end 384. The neck 380 is coupled to the head 352 at the proximal end 382. The neck 380 may connect and/or support the inflator 356 and/or the shaft 304 of the tip 302. In one embodiment, the neck 380 is configured to couple to the shaft 304 of the tip 302. In one embodiment, the neck 380 may be the same, or similar to, the neck 380 implemented in the apparatus 300a.

Advantageously, the apparatus 300b includes a stop 386. The stop 386 may be the same, or similar to, the stop 150 described herein in relation to the system 100 and/or the stop 386 described herein in relation to the apparatus 300a. In certain embodiments, the stop 386 may be one example implementation of a plurality of possible embodiments of the stop 150. Thus, the stop 386 may include one or more or all of the aspects, features, and/or attributes of the stop 150 or stop 386 of apparatus 300a.

In one embodiment, the stop 386 is coupled to the distal end 384 of the neck 380. The stop 386 is configured to limit, stop, prevent, and/or mitigate an insertion distance of the shaft 304 into the ear canal of a patient. The stop 386 prevents over insertion of the tip 302 when the apparatus 300b is in use. The stop 386 may connect or couple to the distal end 384 of the neck 380 in a variety of ways. Examples of these are described in relation to apparatus 300a.

The apparatus 300b includes an inflator 356. The inflator 356 may include one or more, or all. of the aspects, features, and/or attributes of the inflator 112, inflator 356 of apparatus 300a, and/or the inflater 500 described in relation to FIG. 5B. Alternatively, or in addition, the inflator 356 of apparatus 300b may include its own unique features, attributes, and/or aspects.

In the illustrated embodiment, apparatus 300b includes an inflator 356 having a chamber 357 and a driver 364. The chamber 357 includes a fluid 122. In the illustrated embodiment, the chamber 357 houses the fluid 122. Advantageously, the chamber 357 is configured to hold a sufficient quantity of fluid 122 to inflate the balloon 306 as many times as needed during the lifetime of the apparatus 300b. The chamber 357 has a proximal end 358 having a proximal opening 359 and a distal end 360 having a distal opening 361. The distal opening 361 of the chamber 357 can serve as an outlet that provides fluid communication between fluid 122 in the chamber 357 and a passage 305 of the shaft 304.

The driver 364 is configured to force fluid out of the chamber 357 and out the distal opening 361. During use, the distal opening 361 is an outlet for the fluid 122. Also, during use, the distal opening 364 provides fluid communication between fluid 122 in the chamber 357 and a passage 305 of the shaft 304.

In certain embodiments, the 364 is configured to pressurize the chamber 357 such that fluid 122 in the chamber 357 flows through the passage 305 and into the balloon 306. Those of skill in the art will appreciate that the driver 364 can have a variety of configurations and implementations. In the illustrated embodiment, the driver 364 includes a piston 365 that includes a rod 366, a plunger seal 367, and a cap 368.

In this example, the piston 365 is manually operated. The piston 365 serves to pressurize the chamber 357. The rod 366 extends into the body 350 and at least partially into the chamber 357. The rod 366 also extends proximally from the body 350. The rod 366 may extend such that at least a portion of the rod 366 extends outside the body 350.

The plunger seal 367 serves to provide a leak proof seal of an opening of the chamber 357 that holds the fluid 122. The plunger seal 367 may block and/or extend at least partially into the chamber 357. In this manner, the plunger seal 367 is within the chamber 357. In certain embodiments, the plunger seal 367 is coupled to the rod 366 such that movement of the rod 366 moves the plunger seal 367 within the chamber 357. In one embodiment, movement of the rod 366 and plunger seal 367 from the proximal end 358 of the chamber 357 towards the distal end 360 of the chamber 357 drives fluid 122 through the passage 305 and into the balloon 306.

In another embodiment, the chamber 357 may include a combination of a liquid fluid and a gas fluid configured such that liquid fluid fills the chamber 357 towards the proximal end 358 and gas fluid occupies the balloon 306 (when deflated) and the passage 305 and may occupy a portion of the chamber 357 towards the distal end 360. In such an embodiment, movement of the rod 366 towards the tip 302 drives the liquid fluid toward the gas fluid and the gas fluid may inflate the balloon 306.

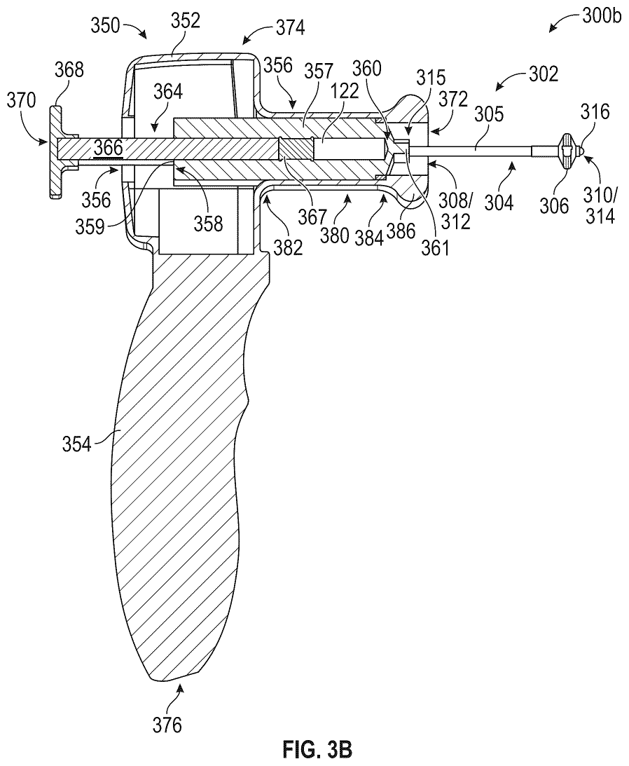

FIGS. 4A-4B are perspective views of parts of an apparatus 400 for removing cerumen from a subject or patient according to one embodiment. The apparatus 400 may be substantially the same, or similar to, the apparatus 300a and/or the apparatus 300b described herein. In FIGS. 4A and 4B, the tip 302 and inflator 356 is omitted.

FIGS. 4A-4B are perspective views of a body 350 according to one embodiment. FIG. 4A is an anterior perspective view of the body 350 and FIG. 4B is a posterior perspective view of the body 350. The body 350 may have many structures, features, and functions, operations, and/or configuration similar or identical to those of the body 350 described in relation to FIG. 3A and/or FIG. 3B, like parts are identified with the same or similar reference numerals. Accordingly, the body 350 may include a head 352, a handle 354, a neck 380, a stop 386 and have a superior end 374 and an inferior end 376.

FIGS. 4A-4B illustrate specific features and/or aspects of a handle 140, such as handle 354, according to one embodiment. Advantageously, the handle 354 includes a grip 402. In certain embodiments, the grip 402 is on an anterior surface of the handle 354. In another embodiment, the grip 402 is on a posterior surface of the handle 354. In another embodiment, the grip 402 is on an anterior surface, a posterior surface, and on each side surface of the handle 354.

The grip 402 facilitates handling and manipulation of the apparatus 400 by a user, subject, or patient. In one embodiment, the grip 402 is configured to accommodate one or more fingers of a user. By engaging the handle 354 with one or more fingers, a user has superior control and management of the apparatus 400.

The grip 402 can have a variety of features, aspects, and/or configurations. In the illustrated embodiment, the grip 402 includes a palm grip 404, at least two or more finger grips 406 (i.e., finger grip 406a, finger grip 406b, finger grip 406c, finger grip 406d) and an optional thumb grip 408. In certain embodiments, the grip 402 is configured to enable a user to comfortably grasp the handle 354. In certain embodiments, the handle 354 and/or grip 402 is configured for a target user. For example, one size handle 354 and/or grip 402 may be configured for adult users/patients and another size handle 354 and/or grip 402 may be configured for pediatric users/patients.

The grip 402 may be shaped and/or configured using one or more ridges and/or depressions, indentations, and/or voids on, or about, one or more surfaces of the handle 354. In certain embodiments, the grip 402 may include a plurality of extensions, protrusions, indentations, curves, rounded sections, carveouts, and/or the like. The grip 402 can be ergonomic. In certain embodiments, the grip 402 is configured to receive at least four fingers of a user holding the handle 354.

Referring now to FIG. 4B, the palm grip 404 is configured to accommodate and/or engage with a palm of a hand of a user as the user uses the apparatus 400. In the illustrated embodiment, the palm grip 404 may be implemented by a raised curved semi-circular extension on a posterior surface of the handle 354. The palm grip 404 may be textured with a smooth texture to fit and engage comfortably in the palm of a user. In certain embodiments, the palm grip 404 may include a soft, flexible, and/or compressible material such as foam, nylon, rubber, or the like.

In various embodiments, the present aspect pertains to a palm grip 404 incorporated into the design of a handle for an apparatus 400. The palm grip 404 may be specifically engineered to provide users with a secure, comfortable, and ergonomic grip during the operation of the medical device, potentially enhancing user control, stability, and safety.

Surface Area and Contoured Shape: The handle 354 of the apparatus 400 may be constructed to offer ample surface area to potentially accommodate the entire palm of the user's hand. Furthermore, the handle 354 may feature a contoured or ergonomic shape that could conform to the natural curvature of the palm. This design consideration may promote a comfortable grip and potentially minimize the risk of slippage or discomfort during prolonged use.

Texture or Gripping Material: In certain embodiments, the surface of the handle 354 might be enhanced with texture or a gripping material, such as rubber or silicone. This textured surface could provide additional traction and potentially ensure a secure grip, even when the user's palm becomes moist or sweaty. The incorporation of such materials might further enhance user control and potentially mitigate the potential for accidents or mishandling of the apparatus 400.

Supportive Structure: The handle 354 may be structurally designed to potentially provide adequate support for the hand, thereby potentially reducing strain and fatigue associated with prolonged device operation. By distributing pressure evenly across the palm and wrist, the palm grip feature might facilitate comfortable and efficient handling of the medical device, particularly during critical or time-sensitive procedures.

Accessibility and Usability: The palm grip 404 may ensure accessibility and ease of use for users across various medical settings. The design and/or configuration might allow for intuitive grasping and manipulation of the handle 354, enabling users to potentially maintain precise control and maneuverability during complex procedures. (© ChatGPT 3.5 Version, Modified, accessed chat.openai.com/chat around Apr. 9, 2024).

Alternatively, or in addition, the grip 402 may include one or more finger grips 406. In the illustrated embodiment, the grip 402 includes at least three finger grips 406a-c each configured to accommodate and/or engage with one or more fingers of the hand of a user as the user uses the apparatus 400.

In certain embodiments, the finger grips 406 (e.g., finger grips 406a-d) may be part of a single finger grip section configured to receive and/or accommodate a two or more fingers of a user. For example, in the illustrated embodiment, the finger grips 406 may include a set of two finger grips 406. Finger grip 406a may be configured to receive a single finger such as an index or pointer finger and finger grips 406b-d may be configured to receive a plurality of fingers such as a middle finger, a ring finger, and a pinky finger. In such an embodiment, a ridge 410 may separate the two finger grips 406.

In one embodiment, the finger grips 406 (e.g., finger grips 406a-d) may each be configured to receive and/or accommodate a particular finger of a user. For example, finger grip 406a may be size and/or include a contour for receiving an index or pointer finger of the user. Finger grip 406b may be size and/or include a contour for receiving a middle finger of the user. Finger grip 406c may be size and/or include a contour for receiving a ring finger of the user. Finger grip 406d may be size and/or include a contour for receiving a little or pinky finger of the user. In such embodiments, a user grasping the handle 354 will feel their respective fingers fit within the respective finger grips 406.

In certain embodiments, the grip 402 may also include a thumb grip 408. The thumb grip 408 is configured to accommodate and engage with a thumb of the hand of the user as the user handles the apparatus. A user may rest their thumb on the thumb grip 408 as the user carries or manipulates the apparatus 400. In certain embodiments, the user may place their thumb temporarily on the thumb grip 408 until they are prepared to inflate the balloon 306. In one embodiment, a user may place their thumb on the cap 368 (See FIG. 3B) as the user inflates the balloon 306 and/or uses the apparatus 300b, 400.

FIG. 5A is a side view of an inflator 500 according to one embodiment. The inflator 500 may be the same, or similar to, the inflator 112 and/or inflator 356 described herein in relation to the system 100 and/or apparatus 300a and/or apparatus 300b. In certain embodiments, the inflator 500 may be one example implementation of a plurality of possible embodiments of the inflator 112 and/or inflator 356. Thus, the inflator 500 may include one or more or all of the aspects, features, and/or attributes of the inflator 112 and/or inflator 356. FIG. 5B is an exploded view of an inflator 500 implemented as a syringe 510 according to one embodiment.

In certain embodiments, the inflator 500 can be installed and/or used in an embodiment of apparatus 400 (See FIGS. 4A, 4B) that can be used for removing cerumen from a subject. Apparatus 300b illustrates one example of such a combination.

In one embodiment, the inflator 500 may be embodied as a syringe 510. In the illustrated embodiment, the syringe 510 is configured to include, connect to, or is connectable to, a tip that is the same and/or similar to the tip 302 described in relation to apparatus 300a or apparatus 300b. The syringe 510 may be configured to connect to a tip 302 instead of a needle. The tip 302 includes a balloon 306.

Combining the apparatus 400 and the inflator 500 can provide an apparatus that is the same and/or similar to the apparatus 300b. In such an embodiment, the apparatus 300b/400 includes a tip 302 having a balloon 306 and a shaft 304 coupled to the balloon 306. The shaft 304 includes a passage 305 that is in fluid communication with an interior surface of the balloon 306. The syringe 510 is configured to couple to a proximal end 312 of the shaft 304 (See FIG. 3B) by way of a coupling 315. The syringe 510 is configured to drive a fluid 122 into the passage 305. The body 350 includes and/or secures the syringe 510 and/or positions the syringe 510 such that the shaft 304 extends away from the body 350. In one embodiment, the shaft 304 extends distally away from the body 350. The handle 354 connects to the body 350.

Referring to FIGS. 5A and 5B, the syringe 510 includes a barrel 512 and an actuator 514. The barrel 512 may be referred to as a chamber in certain embodiments. The barrel 512 includes a fluid 122. In the illustrated embodiment, the barrel 512 houses the fluid 122. Advantageously, the barrel 512 is configured to hold a sufficient quantity of fluid 122 to inflate the balloon 306 as many times as needed during the lifetime of the system 100, apparatus 300a, apparatus 300b, apparatus 400, and/or inflator 500. The barrel 512 has a proximal end 516 having a proximal opening 518 and a distal end 520 having a distal opening 522.

The syringe 510 may include a driver 524 configured to force fluid out of an inner cavity or chamber of the barrel 512 and out the distal opening 522. During use, the distal opening 522 is an outlet for an internal chamber such as an internal chamber of the barrel 512. Also, during use, the outlet or distal opening 522 provides fluid communication between fluid 122 in the chamber and a passage 526 of the shaft 304. In one embodiment, the distal opening 522 is configured to interface with the passage 526 to enable fluid 122 to flow between the barrel 512 and the passage 526 in response to operation of an actuator 514 and/or driver 524. In certain embodiments, fluid 122 can flow both out of, and into, the barrel 512 by way of the distal opening 522. In another embodiment, fluid 122 may flow out of the barrel 512 but not into the barrel 512 by way of the distal opening 522.

In certain embodiments, the diameter of the distal opening 522 may be slightly larger than the outer diameter of the proximal end 312 of the shaft 304. In this manner, the proximal end 312 may be coupled to the distal opening 522 of the barrel 512 by way of a friction or slip fit. Alternatively, or in addition, the distal opening 522 and proximal end 312 may be coupled by a coupler, such as a Luer lock, for example.

The driver 524 may be the same, or similar to, the driver 110, 110a, 110b described herein in relation to the system 100. In certain embodiments, the driver 524 may be one example implementation of a plurality of possible embodiments of the driver 110, 110a, 110b. Thus, the driver 524 may include one or more or all of the aspects, features, and/or attributes of the driver 110, 110a, 110b.

In the illustrated embodiment, the driver 524 may be implemented by an actuator 514 that operates in conjunction with the barrel 512 to move fluid 122 out of, or into, the distal opening 522. In particular, the driver 524 is configured to pressurize a chamber, or an internal space, within the barrel 512. The pressure added to the chamber by the driver 524 moves fluid 122 and causes the fluid 122 to flow out the distal opening 522 and into a passage 526 of the shaft 304.

In certain embodiments, the actuator 514 and/or driver 524 may include a piston 528. The piston 528 may be implemented as a plunger 530. The plunger 530 may include a rod 532, a plunger seal 534, and a knob 536. The plunger 530 is configured such that the plunger seal 534 sits within a chamber or open space of the barrel 512 near the proximal end 516 and distal to the proximal opening 518 of the barrel 512. The plunger 530 may extend proximally from the body 350. The plunger 530 is configured to move laterally within the barrel 512 from the proximal end 516 towards the distal end 520 and from the distal end 520 towards the proximal end 516.

The plunger seal 534 is coupled to the rod 532 such that movement of the rod 532 moves the plunger seal 534 within the chamber. Specifically, movement of the rod 532 toward the chamber drives fluid 122 into the passage 526.

In one embodiment, the plunger seal 534 comprises a single seal. In another embodiment, the plunger seal 534 comprises two or more seals. In the illustrated embodiment, the plunger 530, plunger seal 534, and/or chamber may be coaxial along a longitudinal axis and may have a circular cross section. The plunger seal 534 may be coupled to the rod 532 near a distal end of the rod 532. In certain embodiments, the plunger seal 534 has a diameter slightly larger than a diameter of the proximal opening 518 and/or a chamber within the barrel 512. In this manner, fluid 122 distal to the distal end of the rod 532 does not leak past the plunger seal 534 as the plunger 530 operates.

The rod 532 may be include a knob 536 connected, or coupled, near, or at, a proximal end of the rod 532. The knob 536 may be used by a user to drive the rod 532 towards the chamber (into a syringe). In one embodiment, the knob 536 has a circular cross section and a larger diameter than the rod 532 to facilitate a user pressing the plunger 530. In certain embodiments, a user may grasp the knob 536 between an index finger and a middle finger and their thumb and pull on the knob 536 to move the rod 532 away from the chamber, for example to extract fluid 122 from the passage 526 and/or balloon 306.

FIG. 5A illustrates that the plunger 530 extends from a proximal opening 518 in the barrel 512 (e.g., chamber) and part of the plunger 530 (e.g., plunger seal 534) is within the barrel 512. In the illustrated embodiment, the plunger 530 together with the barrel 512 can serve as an actuator.

FIG. 5B illustrates an exploded view of one embodiment of an inflator 500. To assemble the inflator 500, the plunger seal 534 is inserted into the barrel 512 by way of the proximal opening 518. The shaft 304 is coupled to the distal end 520 of the barrel 512 by way of the distal opening 522 and/or a coupler. The balloon 306 is connected to shaft 304 near a distal end of the shaft 304.

As the inflator 500 pressurizes fluid 122 within the shaft 304, the fluid flows through the passage 526 and into the balloon 306. In one embodiment, the balloon 306 has a balloon cross-section diameter that increases as fluid and/or fluid pressure within the balloon 306 increases. Advantageously, with the balloon 306 inserted at least partially into an auditory canal of a patient, increasing the balloon cross-section diameter causes the balloon 306 to engage with a surface of the auditory canal. The increasing balloon cross-section diameter is caused by at least partial inflation of the balloon 306 by the fluid 122 driven by the inflator 500, which can be a syringe 510. Engagement of the balloon 306 with the surface of the auditory canal provides an effective mechanism for extracting cerumen, because the balloon 306 can be inflated to a balloon cross-section diameter such that the balloon presses against the surface of the auditory canal and can then scrape cerumen from the surface of the auditory canal as the balloon 306 is extracted from the auditory canal.

In the illustrated embodiment, the shaft 304 includes an opening 538. In one embodiment, the opening 538 is on one side of the shaft 304 near a distal end of the shaft 304. Alternatively, or in addition, the shaft 304 may include a plurality of openings 538. The opening 538 provides a path for fluid from the passage 526 of the shaft 304 and into an interior of the balloon 306. In certain embodiments, the shaft 304 includes a single opening on a proximal end to the passage 526 and a single opening 538. Alternatively, or in addition, the shaft 304 may also include an opening on a distal end of the shaft 304. An opening on the distal end of the shaft 304 may be closed by a cap or otherwise closed prior to use and/or assembly of the inflator 500.

In an alternative embodiment, the inflator may include a chamber that contains fluid 122. The chamber may also be referred to as a bladder or bellows. The chamber may be three-dimensional and may include an outlet similar to the distal opening 522 of inflator 500 in FIGS. 5A, 5B. This outlet may be coupled to a passage such as passage 526 of a shaft 304. In this embodiment, one wall of the chamber (such as a proximal wall) may be a flexible, pliable, and/or deformable wall (i.e., flexible wall).

The alternative embodiment may include a driver implemented as a compression force that can be applied to the one wall of the chamber. For example, the flexible wall may be accessible to a user, for example through an opening or the flexible wall may be part of an external surface for an earwax removal device. In one embodiment, a user may press their thumb and/or one or more fingers against the flexible wall. The force from a user's finger(s) creates a compression force that reduces a volume of the chamber such that fluid 122 inside the chamber is driven into the passage 526.

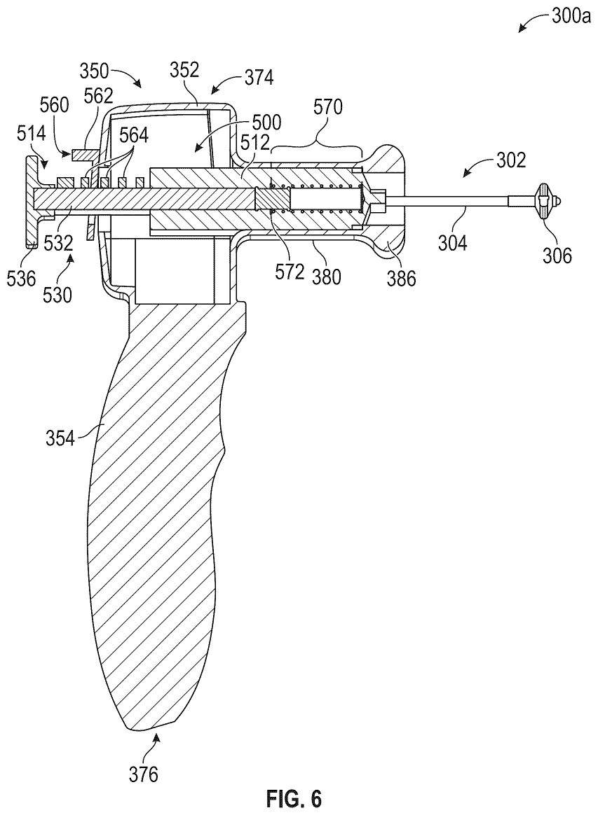

FIG. 6 is a cross-section view illustrating a view along lines 3B/6-3B/6 of FIG. 3A, according to one embodiment. As with other figures herein, FIG. 6 may include one or more parts, structures, or components as those illustrated in relation to other embodiments. Like parts are identified with the same or similar reference numerals.

FIG. 6 illustrates some of the internal workings of one embodiment, such as apparatus 300a. In the illustrated embodiment, the apparatus 300a includes a driver implemented using an inflator 500 and an actuator 514. The actuator 514 extends proximally from the body 350. Specifically, in the illustrated embodiment, the inflator 500 includes a rod 532 that extends proximally from the body 350.

FIG. 6 illustrates how a user may use the apparatus 300a or apparatus 300b. A user may grasp the handle 354, position the tip 302 in an ear canal, and then depress the actuator 514 by pressing on the knob 536. This action drives fluid 122 from the barrel 512 into the passage 526 of the shaft 304. The fluid 122 flows through the shaft 304 and into the balloon 306 to inflate the balloon 306. In one embodiment, pressing the rod 532 towards the tip 302 activates the inflator 500 to inflate the balloon 306.

Next, a user may maintain pressure on the knob 536 to keep the balloon 306 inflated as the user extracts the tip 302 from the ear canal. As the balloon 306 is extracted of a patient's ear canal, the increased diameter of the inflated balloon 306 causes the balloon 306 to engage with one or more walls of the ear canal and push cerumen towards an external opening of the ear canal (e.g., the distal end of the ear canal).

In certain embodiments, pressing the rod 532 into the barrel 512 may inflate the balloon 306 and the rod 532 may remain in a pressed position without the user maintaining pressure or continuing to hold the knob 536. Alternatively, or in addition, the apparatus 300a may include additional features to facilitate use of the apparatus 300a. For example, the apparatus 300a may also include a lock 560 and a biasing member 570.

In one embodiment, the lock 560 is configured to maintain an inflation state of the balloon 306. In certain embodiments, the lock 560 may provide this function by stopping a decrease in pressure of fluid 122 within the balloon 306. Alternatively, or in addition, the lock 560 may cooperate with a driver to maintain an inflation state of the balloon 306.

In the illustrated embodiment, the lock 560 is configured to secure a plunger 530 (i.e., the rod 532) in a depressed state. A depressed state is one in which the plunger 530 has been pressed into the inflator 500. The lock 560 may include a latch 562 and one or more catches 564. The latch 562 may be a slidable structure that a user can slide into place to lock the plunger 530 in place. The lock 560 may include a single catch 564 or a plurality of one or more catches 564. The catches 564 engage with the latch 562 and retain, stop, and/or prevent lateral movement of the plunger 530.

The biasing member 570 may serve to return the plunger 530 to an original state or position after being depressed. Thus, the biasing member 570 may provide for automatic deflation of a balloon 130 which facilitate use, reuse, and practicality of the apparatus 300a.

In one embodiment, the biasing member 570 may be implemented as a coil spring. The biasing member 570 may be coupled to the plunger 530 may engage or contact a flange 572 or lip 572 coupled to the plunger 530 (i.e., near a distal end of the plunger 530).

The biasing member 570 may be coaxial with a longitudinal axis of the plunger 530. The biasing member 570 may be positioned and/or configured such that depressing the plunger 530 creates or stores a biasing force that can act on the plunger 530 in a direction opposite to a direction the plunger 530 moves when being depressed.

In one embodiment, the biasing member 570 is configured to provide sufficient opposite biasing force that the plunger 530 can be readily depressed by a user and still move the plunger 530 in the direction opposite the depressing direction, when the depressing force stops and/or when the plunger 530 is free to move laterally. Said another way, the biasing force of the biasing member 570 is designed to not impede pressing the plunger 530 to inflate the balloon 306 but can also retract the plunger 530 to deflate the balloon 306 when needed.

In certain embodiments, the apparatus 300a is a closed fluid system. Consequently, pressing the plunger 530 inflates the balloon 306 and retracting the plunger 530 draws fluid 122 from the balloon 306 and deflates the balloon 306. In this manner, the state of the balloon 306 can readily be changed for the cerumen extraction operation. When the balloon 306 is inside an ear canal the balloon 306 can be inflated, and when the balloon 306 is removed from the ear canal the balloon 306 can be readily deflated.

In certain embodiments, such as an alternative embodiment, the tip 302 may include a plurality of balloons 306. For example, in one embodiment, the tip 302 includes a distal balloon 306 that is closer to a distal end of the shaft 304 and a proximal balloon that is between the distal balloon 306 and a proximal end of the shaft 304. In such an embodiment, the proximal balloon may have a smaller diameter when inflated than the distal balloon 306. This configuration may be advantageous in that the proximal balloon may engage and contact a top layer of cerumen and/or assist in freeing cerumen, while the distal balloon 306 may then engage with remaining cerumen on walls of the ear canal to provide enhanced extraction capacity. In certain embodiments, a different type of balloon 306 may be used with a different type of cerumen in the ear canal. In another embodiment, a tip 302 may not include a balloon 306 and instead may include one or more soft rubber structures that serve as scratchers to scratch walls of an ear canal to relieve a patient of an ear itch.

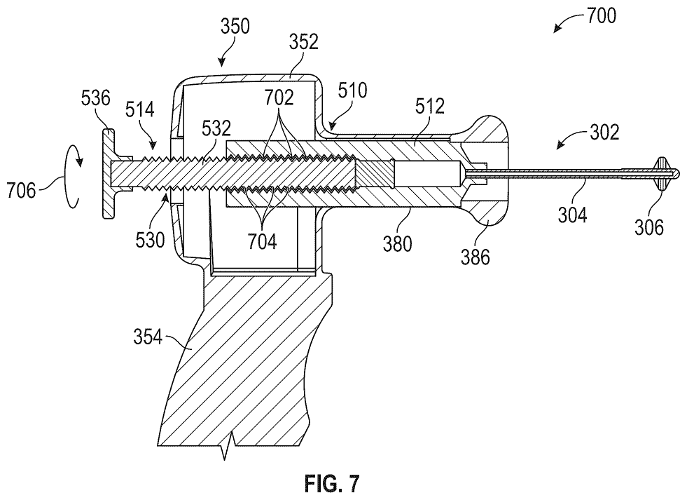

FIG. 7 is a cross-section view illustrating a view along lines 3B/6-3B/6 of FIG. 3A, according to one embodiment. FIG. 7 illustrates an alternative embodiment of an apparatus. In the apparatus 700 of FIG. 7, a different mechanism is used to operate and move the plunger 530. FIG. 7 illustrates an embodiment in which the neck 380 supports the syringe 510, similar to FIGS. 3B, 6.

In the illustrated embodiment, the plunger 530 is coaxial with a longitudinal axis of the syringe 510 and barrel 512. In this embodiment, an internal chamber of the barrel 512 may include internal threads 702 and an external surface along at least a portion of the rod 532 may include external threads 704. The external threads 704 are configured to correspond to and screw into the internal threads 702. The threads 702/704 may be clockwise or counterclockwise threads.

The threads 702/704 provide an alternative mechanism for driving the rod 532 into the barrel 512 and for retracting the rod 532 from the barrel 512. In this example, the plunger 530 engages a proximal opening 518 of the barrel 512. The threads 702/704 include at least one screw thread that extends along an external surface of at least part of the plunger 530.

In this embodiment, a user may rotate the rod 532, by for example, turning the knob 536 in the direction of arrow 706 (e.g., clockwise). As the rod 532 rotates, the rod 532 moves deeper into the barrel 512 and thus pushes fluid 122 into the balloon 306. Similarly, as a user rotates the knob 536 in an opposite direction (e.g., counterclockwise), the threads 702/704 move past each other and the rod 532 moves out of the barrel 512 which draws fluid 122 out of the balloon 306 and deflates the balloon 306.

Of course, those of skill in the art will appreciate that various mechanisms may be used for activating an inflator and inflating and/or deflating the balloon. For example, in one embodiment, the handle 354 may be configured to include a trigger linked to the rod 532 such that pulling the trigger causes the rod 532 to move and inflate the balloon 306. Similarly, releasing the trigger causes the rod 532 to retract and deflate the balloon 306. In one embodiment of such an example that include a trigger linked to the rod 532, the trigger mechanism may be configured such that activating the trigger causes a slow and/or controlled movement of the rod 532 which causes inflation of the balloon 306. In this manner, the slow movement of the rod 532 and/or inflation of the balloon 306 can mitigate a risk of bursting the balloon 306 and/or startling the user/patient.

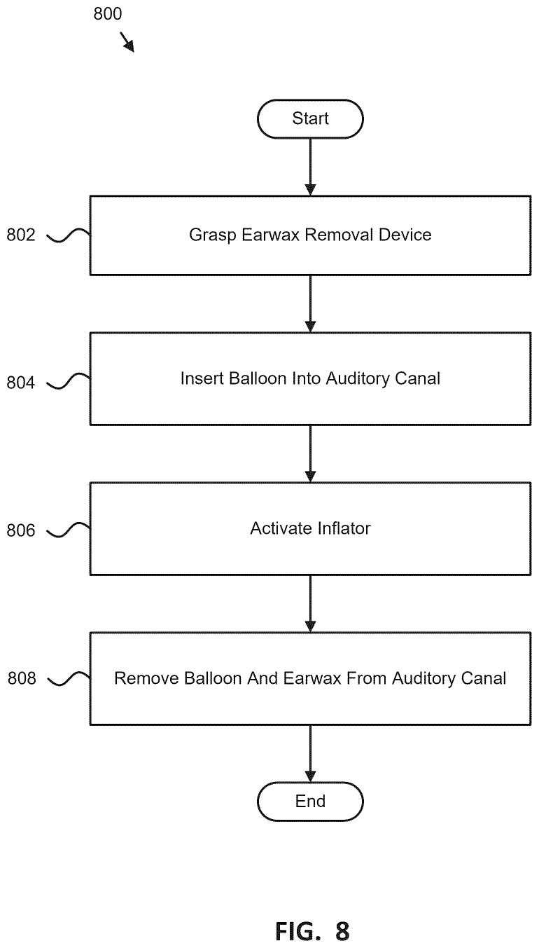

FIG. 8 is a flowchart of an example method 800 for removing cerumen, according to one embodiment. In some implementations, one or more method steps of FIG. 8 may be performed by a system 100, apparatus 300a, apparatus 300b, apparatus 700, device, or the like.

As shown in FIG. 8, method 800 may include grasping an earwax removal device (e.g., system 100, apparatus 300a, apparatus 300b, apparatus 700) having: a tip that includes a flexible shaft having a passage that extends from a proximal end of the flexible shaft; a balloon coupled near a distal end of the flexible shaft, the balloon in fluid communication with the passage (step 802).

As also shown in FIG. 8, the earwax removal device may include a body having: a head coupled to a neck that is configured to couple to the flexible shaft of the tip; a handle coupled to the head; an inflator coupled to the flexible shaft and configured to inflate the balloon by forcing a fluid through the passage; and a stop coupled to the distal end of the neck, the stop configured to prevent contact between the distal end of the flexible shaft (or a balloon 306 coupled to the shaft) and a tympanic membrane of a patient. In one embodiment, the handle 140 or handle 354 is configured for convenient, safe, and ergonomic use in either a left hand or a right hand of a user.

As further shown in FIG. 8, method 800 may include inserting the balloon into an auditory canal of a patient until the stop contacts an ear of the patient (step 804). In one embodiment, the stop contacts one of a tragus and/or an antitragus of an ear.

As also shown in FIG. 8, method 800 may include activating the inflator to inflate the balloon. In one embodiment, a user may activate the inflator by way of a thumb of a user pressing a plunger to inflate the balloon (step 806).

As further shown in FIG. 8, method 800 may include removing the balloon from the auditory canal, the inflated balloon pushing cerumen toward a proximal opening of the auditory canal (step 808).

Method 800 may include additional implementations, such as any single implementation or any combination of implementations described below and/or in connection with one or more other methods or processes described elsewhere herein.

In one implementation, the method 800 may include exchanging the tip with another tip configured to remediate a condition of an ear of a patient. Examples of alternative tips include, a tip with a shaft 304 and a medicine, drug, salve, or the like within a fluid supply 120. The shaft 304 may include the same opening 538 as in prior embodiments. Alternatively, or in addition, the shaft 304 may include an opening at or near the distal end of the shaft 304 such that activation of the driver 110 or driver 524 deploys at least some of the contents of the fluid supply 120 in the ear canal.

In one embodiment, a user may clean the balloon and/or shaft 304 of cerumen before a subsequent use of the earwax removal device. In another embodiment, a tip 302 is exchanged for another clean or unused tip 302 for another cerumen extraction procedure on the same ear canal or on another ear of the patient or on an ear of another patient.

In another implementation, alone or in combination with the first implementation, the user is the subject and/or the patient. This means that the patient is self-administering a procedure for cerumen removal from their own ears. This feature can provide cost savings and improved hygiene and health for the user.

A another implementation, alone or in combination with the first and second implementation, method 800 further includes deflating the balloon; removing extracted cerumen from the tip (or cleaning and/or disinfecting the tip 302); inserting the balloon into an auditory canal of a patient until the stop contacts one of a tragus and an antitragus of an ear of the patient; activating the inflator by way of the thumb of the user to inflate the balloon; and removing the balloon from the auditory canal, the balloon in an inflated state such that the inflated balloon scrapes cerumen out of the auditory canal.

Those of skill in the art will appreciate that the method 800 can include reuse of the same tip in the same ear of a user, in another ear of the user, or in an ear of another user or patient. Alternatively, or in addition, each time the earwax removal device is used on a different ear (of the same subject/user or another subject/user) a different tip may be attached to the earwax removal device. The different tip may be a new unused tip or a cleaned and/or disinfected tip.

Although FIG. 8 shows example steps of method 800, in some implementations, method 800 may include additional steps, fewer steps, different steps, or differently arranged steps than those depicted in FIG. 8. Additionally, or alternatively, two or more of the steps of method 800 may be performed in parallel.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. An apparatus for removing cerumen from a subject, the apparatus comprising:
a tip comprising:
a shaft comprising a passage that extends from a proximal end of the shaft;
a balloon coupled to the shaft away from the proximal end and coupled to the shaft such that the balloon is in fluid communication with the passage;
a body comprising:
a head configured to couple to the shaft of the tip;
a handle coupled to the head; and
an inflator in fluid communication with the shaft and configured to inflate the balloon by forcing a fluid through the passage.

2. The apparatus of claim 1, wherein the proximal end of the shaft is a proximal end of the tip configured to couple the tip to the body by way of a coupling such that the tip is replaceable.

3. The apparatus of claim 1, wherein the coupling comprises a luer lock.

4. The apparatus of claim 1, wherein the body comprises a neck having a proximal end coupled to the head and a distal end configured to couple to the shaft of the tip.

5. The apparatus of claim 1, wherein the handle comprises a grip configured to accommodate one or more fingers of a user, the grip comprising:
a palm grip configured to accommodate and engage with a palm of a hand of a user as the user uses the apparatus; and
at least two finger grips configured to accommodate and engage with the fingers of the hand of the user as the user uses the apparatus.

6. The apparatus of claim 5, wherein the grip comprises:
a thumb grip to accommodate and engage with a thumb of the hand of the user as the user handles the apparatus.

7. The apparatus of claim 4, wherein the body comprises a stop coupled to the distal end of the neck, the stop configured to limit an insertion distance of the shaft into an ear canal of a subject.

8. The apparatus of claim 1, wherein the inflator comprises:
a chamber comprising the fluid, the chamber comprising an outlet that provides fluid communication between fluid in the chamber and the passage of the shaft; and
a driver configured to pressurize the chamber such that fluid in the chamber flows through the passage and into the balloon.

9. The apparatus of claim 8, wherein the driver comprises a piston comprising:
a rod that extends proximally from the body; and
a plunger seal within the chamber, the plunger seal coupled to the rod such that movement of the rod moves the plunger seal within the chamber and drives fluid through the passage and into the balloon.

10. The apparatus of claim 1, further comprising an actuator configured to activate the inflator to inflate the balloon and wherein the inflator comprises a syringe comprising:
a barrel comprising the fluid;
wherein the barrel comprises a distal end having a distal opening and a proximal end having a proximal opening;
wherein the distal opening is configured to interface with the passage to enable fluid to flow between the barrel and the passage in response to operation of the actuator; and
wherein the actuator comprises a plunger that extends from the proximal opening of the barrel and extends proximally from the body.

11. The apparatus of claim 10, wherein the plunger engages the proximal opening by way of a screw thread that extends along an external surface of at least part of the plunger, and wherein the balloon comprises a textured surface.

12. An apparatus for removing cerumen from a subject, the apparatus comprising:
a balloon;
a shaft coupled to the balloon, the shaft comprising a passage in fluid communication with an interior surface of the balloon;
a syringe configured to couple to a proximal end of the shaft by way of a coupling and configured to drive a fluid into the passage;
a body comprising the syringe, the syringe positioned such that the shaft extends away from the body; and
a handle connected to the body.

13. The apparatus of claim 12, wherein the handle comprises a grip configured to receive at least four fingers of a user holding the handle and wherein the balloon has a balloon cross-section diameter smaller than a canal cross-section diameter of an auditory canal of the subject when the balloon is at least partially deflated.

14. The apparatus of claim 12, wherein the balloon cross-section diameter increases to engage the balloon with a surface of the auditory canal of the subject in response to at least partial inflation of the balloon by the fluid driven by the syringe.

15. The apparatus of claim 12, further comprising a stop coupled to the body such that stop prevents contact of the balloon or the shaft with a tympanic membrane of the subject.

16. The apparatus of claim 12, further comprising a lock configured to maintain an inflation state of the balloon.

17. A method removing cerumen from an ear of a patient comprising:
grasping an earwax removal device comprising:
a tip comprising:
a flexible shaft comprising a passage that extends from a proximal end of the flexible shaft;
a balloon coupled near a distal end of the flexible shaft, the balloon in fluid communication with the passage; and
a body comprising:
a head coupled to a neck that is configured to couple to the flexible shaft of the tip;
a handle coupled to the head;
an inflator coupled to the flexible shaft and configured to inflate the balloon by forcing a fluid through the passage; and
a stop coupled to the distal end of the neck, the stop configured to prevent contact between the distal end of the flexible shaft and a tympanic membrane of a patient;
inserting the balloon into an auditory canal of the patient until the stop contacts an ear of the patient;
activating the inflator to inflate the balloon; and
removing the balloon from the auditory canal, the inflated balloon pushing cerumen toward a proximal opening of the auditory canal.

18. The method of claim 17, further comprising exchanging the tip with another tip configured to remediate a condition of an ear of a patient.

19. The method of claim 17, wherein the user is the subject.

20. The method of claim 17, further comprising:
deflating the balloon;
removing extracted cerumen from the tip;
inserting the balloon into an auditory canal of the patient until the stop contacts one of a tragus and an antitragus of an ear of the patient;
activating the inflator by way of the thumb of the user to inflate the balloon; and
removing the balloon from the auditory canal, the balloon in an inflated state such that the inflated balloon scrapes cerumen out of the auditory canal.
